(19) United States
(12) Patent Application Publication
(10) Pub. No.: US 2021/0388089 A1
(43) Pub. Date: Dec. 16, 2021

(54) ANTIGEN BINDING AGENTS THAT BIND CD277 AND USES THEREOF

(71) Applicants: Jose R. CONEJO-GARCIA, Tampa, FL (US); Kyle Kristopher PAYNE, Philadelphia, PA (US); COMPASS THERAPEUTICS LLC, Cambridge, MA (US); THE WISTAR INSTITUTE OF ANATOMY AND BIOLOGY, Philadelphia, PA (US); MOFFITT CANCER CENTER, Tampa, FL (US)

(72) Inventors: Michael March SCHMIDT, Cambridge, MA (US); Brooke T. MCLAUGHLIN, Cambridge, MA (US); Piotr BOBROWICZ, Cambridge, MA (US); Jose R. CONEJO-GARCIA, Tampa, FL (US); Kyle Kristopher PAYNE, Philadelphia, PA (US)

(21) Appl. No.: 17/267,398

(22) PCT Filed: Aug. 9, 2019
(86) PCT No.: PCT/US2019/046058

§ 371 Date: Feb. 9, 2021

Related U.S. Application Data
(60) Provisional application No. 62/716,551, filed on Aug. 9, 2018.
(60) Provisional application No. 62/846,505, filed on May 10, 2019.
(60) Provisional application No. 62/716,586, filed on Aug. 9, 2018.
(60) Provisional application No. 62/716,636, filed on Aug. 9, 2018.

(51) Int. Cl. C07K 16/28; A61P 35/00; G01N 33/574; G01N 33/50

(52) U.S. Cl. CPC C07K 16/2827; A61P 35/00; G01N 33/57492; G01N 33/505; A61K 2039/505; C07K 2317/71; C07K 2317/565; C07K 2317/24; C07K 2317/21; C07K 2317/72

(57) ABSTRACT

The present disclosure relates to, inter alia, methods for treating, or ameliorating one or more symptoms of, cancer, with compounds (e.g., antibodies, or antigen-binding fragments thereof) that bind to CD277.

### RELATED APPLICATIONS

This application claims the benefit of U.S. Provisional Patent Application Ser. No. 62/846,505 filed on May 10, 2019; U.S. Provisional Patent Application Ser. No. 62/716,586 filed on Aug. 9, 2018; U.S. Provisional Patent Application Ser. No. 62/716,551 filed on Aug. 9, 2018; and U.S. Provisional Patent Application Ser. No. 62/716,636 filed on Aug. 9, 2018. The contents of U.S. Provisional Patent Application Ser. Nos. 62/846,505 and 62/716,586 are herein incorporated by reference in their entireties.

### BACKGROUND

Immune cells, such as T cells, macrophages, and natural killer (NK) cells, can exhibit anti-tumor activity and effectively control the occurrence and growth of malignant tumors. Tumor-specific or -associated antigens can induce immune cells to recognize and eliminate malignancies (Chen & Mellman, (2013) Immunity 39(1):1-10). In spite of the existence of tumor-specific immune responses, malignant tumors often evade or avoid immune attack through a variety of immunomodulatory mechanisms resulting in the failure to control tumor occurrence and progression (Motz & Coukos, (2013) Immunity 39(1):61-73).

T cells play critical roles in effective immune responses by acting as effector cells, influencing B cell production of antibodies, and providing immune memory within the host. T cells can be subdivided into two major subpopulations, αβ T cells and γδ T cells, which reflect the corresponding surface expression of T cell receptors (TCR) αβ and γδ. Among others, αβ T cells recognize non-self-peptide fragments restricted by MHC molecules; γδ T cells recognize unconventional antigens.

γδ T cells are important effectors in an immune response. These T cells lyse pathogen-infected cell and abnormal cells. In addition, these T cells regulate immune responses by inducing dendritic cell (DC) maturation as well as the isotypic switching and immunoglobulin production. This aspect of the immune system is regulated by surface receptors, chemokines and cytokines.

Furthermore, γδ T-cells infiltrate human cancers, but current immunotherapies do not exploit their in situ MHC-independent cytotoxic potential. Modulation of T cell activity may offer opportunities for improved therapies for cancer.

### SUMMARY OF DISCLOSURE

The present disclosure is based, at least in part, upon the discovery that BTN3A1 (butyrophilin subfamily 3 member A1, also referred to herein as CD277) inhibits reactive αβ TCR activation and that anti-CD277 antigen-binding agents, such as the anti-CD277 antibodies described herein can relieve that inhibition, and, thus, are useful for treating cancer, e.g., human cancers. As described herein, the antibodies have anti-tumor efficacy in vivo. Moreover, the antibodies induce or enhance CD277-mediated γδ T cell stimulation. The antibodies also relieve or reduce CD277-mediated inhibition of αβ T cells. Without wishing to be bound by theory, the increased activity of one or both of γδ T cells and αβ T cells enhance anti-tumor immune responses in a subject, resulting in the observed anti-tumor efficacy of the antibodies. Thus, the antibodies (and antigen-binding fragments thereof) described herein are useful for, among other things, treating cancer.

Accordingly, the disclosure provides a method for inducing or enhancing CD277-mediated γδ T cell stimulation in a subject, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal. In some embodiments of these aspects, the CD277-mediated γδ T cell stimulation is CD277-mediated γδ T cell proliferation. In other embodiments of these aspects, the CD277-mediated γδ T cell stimulation is CD277-mediated cytokine production by a γδ T cell. In some embodiments of these aspects, the cytokine production is IFNγ production.

In other aspects, the disclosure provides a method for reducing CD277-mediated inhibition of αβ T cells in a subject, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal. In some embodiments of these aspects, the reduction of CD277-mediated inhibition of αβ T cells is CD277-mediated αβ T cell proliferation. In other embodiments of these aspects, the reduction of CD277-mediated inhibition of αβ T cells is CD277-mediated cytokine production by a αβ T cell. In some embodiments of these aspects, the cytokine production is IFNγ production.

In any of the foregoing aspects and embodiments thereof, the costimulatory signal results from CD3 engagement or CD28 engagement.

In some aspects, the disclosure provides a method for reducing or inhibiting tumor growth, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal.

In other aspects, the disclosure provides a method for treating cancer in a subject, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal.

In yet other aspects, the disclosure provides a method for reducing or inhibiting tumor growth, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal.

In some aspects, the disclosure provides a method for treating cancer in a subject, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal.

In some aspects, the disclosure provides a composition comprising an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal, for use in treating or delaying progression of a cancer, or reducing or inhibiting tumor growth, in a subject in need thereof.

In other aspects, the disclosure provides use of a composition comprising an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal, and a pharmaceutically acceptable carrier, in the manufacture of a medicament for treating or delaying progression of a cancer, or reducing or inhibiting tumor growth, in a subject in need thereof.

In any of the foregoing aspects and embodiments thereof, the antibody or antigen binding portion thereof comprises heavy chain CDR1, CDR2 and CDR3 sequences set forth in SEQ ID NOs: 7-9, respectively, and light chain CDR1, CDR2 and CDR3 sequences set forth in SEQ ID NOs: 10-12, respectively. In any of the foregoing aspects and embodiments thereof, the antibody or antigen binding portion thereof comprises heavy chain CDR1, CDR2 and CDR3 sequences set forth in SEQ ID NOs: 7, 31, and 9, respectively, and light chain CDR1, CDR2 and CDR3 sequences set forth in SEQ ID NOs: 10-12, respectively.

In any of the foregoing aspects and embodiments thereof, the antibody or antigen binding portion thereof comprises heavy and light chain variable regions comprising amino acid sequences set forth in SEQ ID NOs: 3 and 4, respectively. In some embodiments of these aspects and embodiments, the antibody or antigen binding portion thereof comprises heavy and light chain variable regions comprising amino acid sequences at least 90% identical to the amino acid sequences set forth in SEQ ID NOs: 3 and 4, respectively.

In any of the foregoing aspects and embodiments thereof, the antibody or antigen binding portion thereof comprises heavy and light chain variable regions comprising amino acid sequences set forth in SEQ ID NOs: 3 and 34, respectively. In some embodiments of these aspects and embodiments, the antibody or antigen binding portion thereof comprises heavy and light chain variable regions comprising amino acid sequences at least 90% identical to the amino acid sequences set forth in SEQ ID NOs: 3 and 34, respectively.

In any of the foregoing aspects and embodiments thereof, the antibody comprises a heavy chain comprising the amino acid sequence set forth in SEQ ID NO: 26, and a light chain comprising the amino acid sequence set forth in SEQ ID NO: 27.

In other aspects and embodiments thereof, the antibody comprises a heavy chain comprising an amino acid sequence at least 90% identical to SEQ ID NO: 26, and a light chain comprising an amino acid sequence at least 90% identical to SEQ ID NO: 27.

In any of the foregoing aspects and embodiments thereof, the antibody or antigen-binding portion thereof is chimeric or humanized. In some embodiments of these aspects, the antibody or antigen-binding portion thereof is a fully human antibody or antigen-binding portion thereof.

In any of the foregoing aspects and embodiments thereof, the antibody or antigen-binding portion thereof binds to cynomolgus macaque CD277.

In any of the foregoing aspects and embodiments thereof, the antibody is selected from the group consisting of an IgG1, an IgG2, and IgG3, an IgG4, and IgM, and IgA1, and IgA2, and IgD, and an IgE antibody. In some aspects and embodiments thereof, the antibody is an IgG1 antibody or IgG4 antibody.

While the disclosure is not bound by any particular theory or mechanism of action, it is believed that CD277 inhibits tumor-reactive αβ TCR activation by preventing the segregation of CD45 from the immune synapse (e.g., tethering CD45 at the TCR complex). Furthermore, it was discovered that antibody-mediated conformational changes in BTN3A1 rescue αβ T-cell effector activity, while redirecting γδ lymphocytes against BTN3A1+ tumor cells, eliciting coordinated immune rejection of established tumors.

Accordingly, in some aspects, the disclosure provides an antigen-binding agent that specifically binds human CD277, wherein the antigen-binding agent binds to human CD277 and inhibits the interaction of CD277 with CD45, thereby activating or enhancing an αβ T cell response.

In some aspects, the disclosure provides an antigen-binding agent that specifically binds human CD277, wherein the antigen-binding agent binds to human CD277 and inhibits the interaction of CD277 with CD45, thereby disinhibiting the immunosuppressive effect of CD277 towards αβ T cells.

In other aspects, the disclosure provides an antigen-binding agent that specifically binds human CD277, wherein the antigen-binding agent binds to human CD277 and inhibits CD277-mediated association of CD45 with CD3ζ, thereby activating or enhancing an αβ T cell response.

In other aspects, the disclosure provides an antigen-binding agent that specifically binds human CD277, wherein the antigen-binding agent binds to human CD277 and inhibits CD277-mediated association of CD45 with the TCR/MHC immune synapse, thereby activating or enhancing an αβ T cell response.

In other aspects, the disclosure provides an antigen-binding agent that specifically binds human CD277, wherein the antigen-binding agent binds to human CD277 and increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent, or antigen-binding portion thereof, thereby activating or enhancing an αβ T cell response.

In any of the foregoing aspects and embodiments thereof, the antigen-binding agent, or antigen-binding portion thereof, is selected from the group consisting of an antibody, a non-antibody scaffold protein, a polypeptide, a small molecule, and a nucleic acid.

In any of the foregoing aspects and embodiments thereof, the antigen-binding agent, or antigen-binding portion thereof, further induces or enhances γδ T cell response.

In any of the foregoing aspects or embodiments thereof, the TCR signaling molecules are phosphorylated at activating residues LCK^{pY394}, Zap70^{pY319}, and CD3ζ^{pY142}. In some embodiments, the activating residue is LCK^{pY394}. In some embodiments, the activating residue is Zap70^{pY319}. In some embodiments, the activating residue is CD3ζ^{pY142}.

In any of the forgoing aspects and embodiments thereof, the antigen-binding agent is a monoclonal antibody or antigen-binding portion thereof. In some embodiments, the antibody or antigen-binding portion thereof is chimeric or humanized. In some embodiments, the antibody or antigen-binding portion thereof is a fully human antibody or antigen-binding portion thereof. In some embodiments, the antibody is selected from the group consisting of an IgG1, an IgG2, and IgG3, an IgG4, and IgM, and IgA1, and IgA2, and IgD, and an IgE antibody. In some embodiments, the antibody is an IgG1 antibody or IgG4 antibody.

In some aspects, the disclosure provides a composition comprising the monoclonal antibody or antigen-binding portion thereof, as described herein, and a pharmaceutically acceptable carrier.

In some aspects, the disclosure provides a nucleic acid comprising a nucleotide sequence encoding the light chain, heavy chain, or both light and heavy chains of the monoclonal antibody, or antigen-binding portion thereof, as described herein. In further aspects and embodiments thereof, the disclosure provides an expression vector comprising the nucleic acid described herein. In other aspects and embodiments thereof, the disclosure provides a cell transformed with the expression vector described herein.

In some aspects, the disclosure provides a method for treating cancer in a subject, or enhancing a cancer-specific immune response in a subject in need thereof, comprising administering to a subject in need thereof, an effective amount of the antigen-binding agent, or antigen-binding portion thereof, described herein. In some embodiments, the cancer is a solid tumor. In some embodiments, the cancer is ovarian cancer.

In some aspects, the disclosure provides for the use of a composition comprising the antigen-binding agent, or antigen-binding portion thereof, described herein, and a pharmaceutically acceptable carrier, in the manufacture of a medicament for treating or delaying the progression of cancer, or reducing or inhibiting tumor growth in a subject in need thereof. In some embodiments, the cancer is a solid tumor. In other embodiments, the cancer is ovarian cancer.

In some aspects, the disclosure provides a composition comprising the antigen-binding agent, or antigen-binding portion thereof, described herein, for use in treating or delaying progression of a cancer, or reducing or inhibiting tumor growth, in a subject in need thereof. In some embodiments, the cancer is a solid tumor. In other embodiments, the cancer is ovarian cancer.

In other aspects, the disclosure provides a method for identifying an antigen-binding agent of interest, the method comprising: contacting, in the presence of a CD45 protein, a CD277 protein with a test antigen-binding agent; and identifying the test antigen-binding agent as an antigen-binding agent of interest if the test antigen-binding agent inhibits the interaction between CD45 and CD277. In some embodiments, one or both of the CD277 protein and the CD45 protein are recombinant proteins. In other embodiments, one or both of the CD277 protein and the CD45 protein is expressed on the surface of cells. In other embodiments, the CD45 protein is expressed on an αβ T cell.

In other aspects, the disclosure provides a method for identifying an antigen-binding agent of interest, the method comprising: contacting, in the presence of a CD45 protein, a CD277 protein with a test antigen-binding agent, wherein the CD45 protein is expressed by an αβ T cell; and identifying the test antigen-binding agent as an antigen-binding agent of interest if the test antigen-binding agent increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent.

In further aspects, the disclosure provides a method for detecting the immunomodulatory activity of an antigen-binding agent, the method comprising: detecting the presence, absence, or amount of association of: (i) CD45 with CD3ζ or (ii) CD45 with the TCR/MHC immune synapse on one or more αβ T cells from a subject administered an antigen-binding agent described herein, wherein an increase in the association of (i) CD45 with CD3ζ or (ii) CD45 with the TCR/MHC immune synapse on the one or more αβ T cells relative to a control level of association indicates that the antigen-binding agent has immunomodulatory activity.

In some aspects, the disclosure provides a method for detecting the immunomodulatory activity of an antigen-binding agent, the method comprising: detecting the presence, absence, level, or amount of phosphorylation of one or more TCR signaling molecules at activating residues selected from the group consisting of LCK^{pY394}, Zap70^{pY319}, and CD3ζ^{pY142} by one or more αβ T cells from a subject administered an antigen-binding agent described herein, wherein an increase in the level or amount of phosphorylation of one or more TCR signaling molecules at activating residues selected from the group consisting of LCK^{pY394}, Zap70^{pY319}, and CD3ζ^{pY142} by one or more αβ T cells relative to a control level or amount of phosphorylation, indicates that the antigen-binding agent has immunomodulatory activity.

In any of the foregoing aspects and embodiments thereof, one or more T cells is obtained from the subject.

In some aspects, the disclosure provide a method for treating cancer in a subject, the method comprising administering to the subject the antigen-binding agent described herein in an amount effective to treat the cancer, wherein the antigen-binding agent has been determined to have an immunomodulatory effect in the patient. In some embodiments, the immunomodulatory effect was determined as described herein.

In other aspects and embodiments thereof, the antigen-binding agent, the composition, the nucleic acid, the expression vector, the cell, the method, or the use, described herein, comprises an antigen-binding agent comprising heavy chain CDR1, CDR2, and CDR3 sequences set forth in SEQ ID NOs: 7-9, respectively, and light chain CDR1, CDR2, and CDR3 sequences set forth in SEQ ID NOs: 10-12, respectively. In other aspects and embodiments thereof, the antigen-binding agent, the composition, the nucleic acid, the expression vector, the cell, the method, or the use, described herein, comprises an antigen-binding agent comprising heavy chain CDR1, CDR2, and CDR3 sequences set forth in SEQ ID NOs: 7, 31, and 9, respectively, and light chain CDR1, CDR2, and CDR3 sequences set forth in SEQ ID NOs: 10-12, respectively.

In yet other aspects and embodiments thereof, the antigen-binding agent, the composition, the nucleic acid, the expression vector, the cell, the method, or the use, described herein, comprises an antigen-binding agent comprising a heavy chain variable region having at least 90% identity to SEQ ID NO: 3 and a light chain variable region having at least 90% identity to SEQ ID NO: 4. In other aspects and embodiments thereof, the antigen-binding agent, the composition, the nucleic acid, the expression vector, the cell, the method, or the use, described herein, comprises an antigen-binding agent comprising a heavy chain variable region having at least 90% identity to SEQ ID NO: 3 and a light chain variable region having at least 90% identity to SEQ ID NO: 34.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C provide data demonstrating the expression of CD277 in an ovarian tumor microenvironment. FIG. 1A depicts CD1c and CD11c expression on gated CD45+ cells. FIG. 1B depicts CD277 expression on tumor cells, T cells, macrophages, and dendritic cells. FIG. 1C depicts a summary of mean fluorescent intensity (MFI) for CD277 for tumor cells, T cells, macrophages, and dendritic cells.

FIG. 2 depicts expression data for three BTN3A members.

FIG. 3A depicts proliferation fold change data for peripheral blood mononuclear cells (PBMCs) stimulated with BTNA31-K32 cells, and gated for CD4 expression, in the presence of a panel of anti-CD277 antibodies. FIG. 3B depicts proliferation fold change data for PBMCs stimulated with K32 cells, and gated for CD4 expression, in the presence of a panel of anti-CD277 antibodies. FIG. 3C depicts proliferation fold change data for PBMCs stimulated with BTNA31-K32 cells, and gated for CD8 expression, in the presence of a panel of anti-CD277 antibodies. FIG. 3D depicts proliferation fold change data for PBMCs stimulated with K32 cells, and gated for CD8 expression, in the presence of a panel of anti-CD277 antibodies.

FIG. 4A depicts proliferation fold change data for purified CD4+ T cells stimulated with BTNA31-K32 in the presence of a panel of anti-CD277. FIG. 4B depicts proliferation fold change data for purified CD4+ T cells stimulated with K32 cells in the presence of a panel of anti-CD277 antibodies. FIG. 4C depicts proliferation fold change data for purified CD8+ T cells stimulated with BTNA31-K32 cells in the presence of a panel of anti-CD277 antibodies. FIG. 4D depicts proliferation fold change data for purified CD8+ T cells stimulated with K32 cells in the presence of a panel of anti-CD277 antibodies.

FIG. 5A depicts levels of IFN-γ for purified CD4+ T cells stimulated with BTNA31-K32 cells in the presence of a panel of anti-CD277 antibodies. FIG. 5B depicts levels of IFN-γ for purified CD4+ T cells stimulated with K32 cells in the presence of a panel of anti-CD277 antibodies. FIG. 5C depicts levels of IFN-γ for purified CD8+ T cells stimulated with BTNA31-K32 cells in the presence of a panel of anti-CD277 antibodies. FIG. 5D depicts levels of IFN-γ for purified CD8+ T cells stimulated with K32 cells in the presence of a panel of anti-CD277 antibodies.

FIGS. 6A-6D depict T cell proliferation data for purified CD8+ T cells stimulated with BTNA31-K32 cells with and without the presence of zoledronate (FIG. 6A), purified CD8+ T cells stimulated with K32 cells with and without the presence of zoledronate (FIG. 6B), purified CD4+ T cells stimulated with BTNA31-K32 cells with and without the presence of zoledronate (FIG. 6C), and purified CD4+ T cells stimulated with K32 cells with and without the presence of zoledronate (FIG. 6D).

FIGS. 7A-7D depict IFN-γ production data for stimulated CD8+ T and CD4+ T cells. FIG. 7A shows purified CD8+ T cells from two different donors stimulated with BTNA31-K32 cells with and without zoledronate. FIG. 7B shows purified CD8+ T cells stimulated with K32 cells with and without zoledronate. FIG. 7C shows purified CD4+ T cells from two different donors stimulated with BTNA31-K32 cells with and without zoledronate. FIG. 7D shows purified CD4+ T cells stimulated with K32 cells with and without zoledronate.

FIGS. 8A-8B depicts proliferation fold change data for PBMCs stimulated with BTNA31-K32 cells in the presence of a panel of anti-CD277 antibodies without (FIG. 8A) and with (FIG. 8B) zoledronate. FIG. 8C depicts IFN-γ production data for PBMCs under various described conditions.

FIGS. 9A-9B depict cell proliferation data for gated CD4+ T cells (FIG. 9A) and gated CD8+ T cells (FIG. 9B) under experimental conditions described herein.

FIGS. 10A and 10B are graphs showing OVCAR3 tumor volume in mice treated with anti-CD277 antibody mAb1 with or without NY-ESO1 TCR-transduced αβ T cells, or IgG control with NY-ESO1 TCR-transduced αβ T cells from two separate studies. FIG. 10C is a bar graph showing the number of T cells per gram of tissue derived from mice having OVCAR3 tumors 30 days after treatment with anti-CD277 antibody mAb1 with or without NY-ESO1 TCR-transduced αβ T cells, or IgG control with NY-ESO1 TCR-transduced αβ T cells.

FIG. 11A is graph showing OVCAR3 tumor volume in mice that received γδ T cells in addition to anti-CD277 antibody mAb1 or IgG control, or IgG control alone. FIG. 11B is a bar graph showing the percentage of live cells in mice having OVCAR3 tumors that received γδ T cells in addition to anti-CD277 antibody mAb1 or IgG control.

FIG. 12A provides the LC-MS/MS readout of BTN3A1-specific pulldowns after incubation with activated αβ T cells. Data represent 3 independent pull downs from 3 different donors. FIG. 12B Upper panels: depict binding of BTN3A1-Fc protein on the surface of Jurkat cells expressing CD45 (Clone E6-1). BTN3A1-Fc fusion protein did not bind to CD45 negative Jurkat cells (Clone J45.01). Lower panels: depict binding of BTN3A1-Fc protein to the surface of J45.01 cells with forced expression of CD45-RA (lower left panel). Binding of PD-L1-Fc to the surface of J45.01 cells with forced expression of CD45-RA was not observed (lower right panel). FIG. 12C depicts the elimination of BTN3A1 binding in primary αβ T cells with CRISPR ablation of CD45. Binding of BTN3A1 was observed in parental (CD45+) lymphocytes. FIG. 12D Upper panels: depict the segregation of CD45 (red) from CD3ζ (green) in the presence of PD-L1-Fc, but not in the presence of BTN3A1-Fc proteins, after crosslinking the TCR in the presence of plate bound OKT3 and BTN3A1-Fc or control IgG-Fc proteins (all at 10 μg/ml) for 3 minutes. Lower panel: graphically depicts the relative co-localization of CD45 and CD3ζ in the presence of PD-L1-Fc or BTN3A1-Fc. FIG. 12E depicts an immunoblot showing the phosphorylation status of TCR signaling molecules at activating residues in LCK^{pY394}, Zap70^{pY319} and CD3ζ^{pY142} from purified αβ T cells after TCR crosslinking in the presence of plate bound OKT3 and BTN3A1-Fc or control IgG-Fc proteins (all at 10 ug/ml) for 1 minute. *p<0.05, **p<0.01, ***p<0.001.

FIG. 13A graphically depicts the progression of OVCAR3^{NYESO1} tumors in NSG mice treated with NY-ESO-1- or mock-transduced αβ T cells and treated with mAb1 or control IgG every third day (5 mg/kg; i.p.). Left panel: depicts tumor volume, Right panel: depicts tumor weight. FIG. 13B graphically depicts the expression of CD3 and Vγ9 in NY-OVCAR3 tumors. FIG. 13C graphically depicts the absolute number of CD8+ αβ T cells within OVCAR3^{NYESO1} tumors treated with mAb1 or control IgG. FIG. 13D graphically depicts the progression of OVCAR3^{NYESO1} tumors in mice treated with NY-ESO-1- or mock-transduced αβ T cells, and treated with mAb1 or Nivolumab. FIG. 13E graphically depicts PD-L1 expression on OVCAR3 cells. FIG. 13F graphically depicts the progression of OVCAR3^{NYESO1} tumors in NSG mice treated with purified γδ T cells and control IgG, mAb1 alone, or mAb1 and purified γδ T cells, every third day. FIG. 13G depicts the absolute number of γδ T cells within OVCAR3^{NYESO1} tumors treated with mAb1 or control IgG. FIG. 13H depicts the formation of cystic cavities in OVCAR3^{NYESO1} tumors treated with γδ T cells and control IgG or mAb1 and γδ T cells. FIG. 13I graphically depicts the tumor volume of OVCAR3^{NYESO1} tumors in NSG mice treated with mAb1 and γδ T cells, antigen specific T cells and mAb1, antigen specific T cells, γδ T cells, and control IgG, or antigen specific T cells, γδ T cells, and mAb1. The combination of Ag-specific αβ T cells and autologous γδ T cells (ratio of 6:1) with mAb1 is superior in delaying malignant progression of OVCAR3^{NYESO1} tumors. *p<0.05, **p<0.01, ***p<0.001.

FIG. 14A depicts a schematic of the CD11c-BTN3A1 construct. FIG. 14B graphically depicts BTN3A1 expression on BMDCs generated from wildtype C56/BL6 mice, or BTN3A1^{TG} mice. FIG. 14C graphically depicts the proliferation of OT-I T cells in the presence of WT-derived or BTN3A1^{TG}-derived BMDCs previously pulsed with SIITFEKL peptide (black line—wild type; red line—BTN3A1-BDMCs). FIG. 14D graphically depicts the proliferation of OT-I T cells in the presence of BTN3A1^{TG}-derived BMDCs previously pulsed with SIITFEKL peptide and in the presence of mAb1 antibody (right panel) after 72 hrs. FIG. 14E graphically depicts the survival of BTN3A1^{TG} mice bearing ID8-Defb29-Vegf-1 peritoneal tumors treated every 5 days (100 μg, i.p.) with PD-1 neutralizing antibody, mAb1, or control IgG. FIG. 14F graphically depicts the accumulation of CD8+ T cells in the ascites of BTN3A1^{TG} mice bearing ID8-Defb29-Vegf-a peritoneal tumors treated with mAb1 or isotype antibody. *p<0.05, **p<0.01, ***p<0.001. FIG. 14G graphically depicts the Elispot readout comparing IFN-γ release from CD8+ T cells isolated from BTN3A1^{TG} mice bearing ID8-Defb29-Vegf-a peritoneal tumors at day 25, which were treated with mAb1 or isotype control antibody.

### DETAILED DESCRIPTION

### Overview

Various diseases are characterized by the development of progressive immunosuppression in a patient. The presence of an impaired immune response in patients with malignancies has been particularly well-documented. Cancer patients exhibit a variety of altered immune functions such as a decrease in delayed hypersensitivity, and decreases in lytic function and proliferation responses of lymphocytes. Augmenting immune functions in cancer patients may have beneficial effects for tumor control.

In one aspect, the present disclosure provides novel anti-CD277 antibodies. In other aspects, the disclosure provides methods for treating a disorder, such as cancer.

The present disclosure relates to antigen-binding agents that bind CD277 (also referred to herein as BTN3A1), or antigen-binding fragments thereof. The disclosure is based, at least in part, upon the discovery that the butyrophilin BTN3A1 inhibits tumor-reactive αβ TCR activation in human cancer by preventing the segregation of CD45 from the immune synapse. As described in Example 11, it was discovered that BTN3A1 engagement with CD45 prevents the segregation of CD45 away from the TCR:MHC immune synapse, which is required for effective TCR signaling after LCKpY505 dephosphorylation. These data demonstrate that BTN3A1 (CD277) is a negative regulator of αβ TCR signaling.

Furthermore, it was surprisingly discovered that anti-CD277 antibodies targeting BTN3A1 elicit conformational changes in BTN3A1 that transform BTN3A1 from an immunosuppressive to an immunostimulatory molecule. These conformational changes in BTN3A1 overcome the suppressive function of BTN3A1 against αβ T cells, and simultaneously redirect γδ lymphocytes against BTN3A1+ tumor cells, eliciting coordinated immune rejection of established tumors.

These data support a novel mechanism of αβ T-cell suppression and demonstrate that cooperation between αβ and γδ T-cells can be orchestrated against established tumors through BTN3A1 targeting.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise. Further, unless otherwise required by context, singular terms shall include pluralities and plural terms shall include the singular.

As used herein, “αβ T cell” refers to a T cell having a T cell receptor comprised of the highly variable α and β chains, which are expressed in a complex with invariant CD3 chain molecules. An αβ T cell can further be distinguished into “helper T cell” and “cytotoxic T cell” subsets. Helper T cells express the CD4 molecule and play a role in modulating B cell-directed immune responses. Cytotoxic T cells express the CD8 molecule and play an active role in killing damaged cells such as, for example, cancer cells and virally infected cells.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The term “ameliorating” refers to any therapeutically beneficial result in the treatment of a disease state, e.g., cancer, including prophylaxis, lessening in the severity or progression, remission, or cure thereof.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups {e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “angiogenesis” or “neovascularization” refers to the process by which new blood vessels develop from pre-existing vessels (Varner et al., (1999) Angiogen. 3:53-60; Mousa et at, (2000) Angiogen. Stim. Inhib. 35:42-44; Kim et al., (2000) Amer. J. Path. 156:1345-1362; Kim et al., (2000) J. Biol. Chem. 275:33920-33928; Kumar et al. (2000) Angiogenesis: From Molecular to Integrative Pharm. 169-180). Endothelial cells from pre-existing blood vessels or from circulating endothelial stem cells (Takahashi et al., (1995) Nat. Med. 5:434-438; Isner et al., (1999) J. Clin. Invest. 103:1231-1236) become activated to migrate, proliferate, and differentiate into structures with lumens, forming new blood vessels, in response to growth factor or hormonal cues, or hypoxic or ischemic conditions. During ischemia, such as occurs in cancer, the need to increase oxygenation and delivery of nutrients apparently induces the secretion of angiogenic factors by the affected tissue; these factors stimulate new blood vessel formation. Several additional terms are related to angiogenesis.

As used herein, the term “antibody” refers to a whole antibody comprising two light chain polypeptides and two heavy chain polypeptides. Whole antibodies include different antibody isotypes including IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

As used herein, the term “antibody fragment,” “antigen-binding fragment,” “antibody portion,” “antigen-binding portion,” or similar terms refer to a fragment of an antibody that retains the ability to bind to a target antigen (e.g., CD277) and inhibit the activity of the target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al., (2001) J. Immunol. Methods 248(1):47-66; Hudson and Kortt, (1999) J. Immunol. Methods 231(1):177-189; Poljak, (1994) Structure 2(12):1121-1123; Rondon and Marasco, (1997) Annu. Rev. Microbiol. 51:257-283, the disclosures of each of which are incorporated herein by reference in their entirety.

As used herein, the term “antibody fragment” also includes, e.g., single domain antibodies such as camelized single domain antibodies. See, e.g., Muyldermans et al., (2001) Trends Biochem. Sci. 26:230-235; Nuttall et al., (2000) Curr. Pharm. Biotech. 1:253-263; Reichmann et al., (1999) J. Immunol. Meth. 231:25-38; PCT application publication nos. WO 94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed.

In some embodiment, an antigen-binding fragment includes the variable region of a heavy chain polypeptide and the variable region of a light chain polypeptide. In some embodiments, an antigen-binding fragment described herein comprises the CDRs of the light chain and heavy chain polypeptide of an antibody.

An “antigen-binding agent” as described herein can be a single multifunctional polypeptide, small molecule, or aptamer, or it can be a multimeric complex of two or more molecules that are covalently or non-covalently associated with one another. Antigen-binding agents, as described herein, include antibodies (or antigen-binding portions or fragments thereof) that, in some embodiments, can be linked to or co-expressed with another functional molecule, e.g., another peptide, protein, and/or aptamer. The antigen-binding agents described herein can, in various aspects and embodiments, comprise one or more antibodies and/or antigen-binding portions thereof. For example, an antigen-binding unit can comprise a variable heavy and/or variable light chain, or complementarity determining regions thereof, of a given antibody to CD277. The antigen-binding agents described herein can comprise, in part, scaffold domains, proteins, or portions thereof, e.g., molecules which do not provide target receptor-binding activity, but which can provide a portion or domain of the construct which provides spatial organization, structural support, a means of linking of multiple receptor-binding units, or other desired characteristics, e.g., improved half-life. Various scaffold technologies and compositions are known in the art and can be readily linked or conjugated to the antigen-binding units described herein.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

Traditionally, the recombinant production of bispecific antibodies is based on the co-expression of two immunoglobulin heavy-chain/light-chain pairs, where the two heavy chain/light-chain pairs have different specificities (Milstein and Cuello, (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. For further details of illustrative currently known methods for generating bispecific antibodies see, e.g., Suresh et al., (1986) Methods Enzymol. 121:210; PCT Publication No. WO 96/27011; Brennan et al., (1985) Science 229:81; Shalaby et al., J. Exp. Med. (1992) 175:217-225; Kostelny et al., (1992) J. Immunol. 148(5):1547-1553; Hollinger et al., (1993) Proc. Natl. Acad. Sci. USA 90:6444-6448; Gruber et al., (1994) J. Immunol. 152:5368; and Tutt et al., (1991) J. Immunol. 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10):1057-1062. Briefly, these antibodies comprise a pair of tandem Fd segments (VH-CH1-VH-CH1) which form a pair of antigen-binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some embodiments, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

As used herein, the term “cancer” means cells having the capacity for autonomous growth, i.e., an abnormal state or condition characterized by rapidly proliferating cell growth.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The anti-CD277 antibodies described herein can be used to treat patients who have, who are suspected of having, or who may be at high risk for developing any type of cancer, including renal carcinoma or melanoma. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

As used herein, the term antigen “cross-presentation” refers to presentation of exogenous protein antigens to T cells via MHC class I and class II molecules on APCs.

As used herein, the term “CDR” means a complementarity-determining region. One system of CDR numbering is the system described by Kabat, also referred to as “numbered according to Kabat,” “Kabat numbering”, “Kabat definitions”, and “Kabat labeling,” and provides an unambiguous residue numbering system applicable to any variable domain of an antibody, and provides precise residue boundaries defining the three CDRs of each chain. (Kabat et al., Sequences of Proteins of Immunological Interest, National Institutes of Health, Bethesda, Md. (1987) and (1991), the contents of which are incorporated by reference in their entirety. These CDRs are referred to as Kabat CDRs and comprise about residues 24-34 (CDR1), 50-56 (CDR2), and 89-97 (CDR3) in the light chain variable domain, and 31-35 (CDR1), 50-65 (CDR2), and 95-102 (CDR3) in the heavy chain variable domain. When the CDRs are defined according to Kabat, the light chain FR residues are positioned at about residues 1-23 (LCFR1), 35-49 (LCFR2), 57-88 (LCFR3), and 98-107 (LCFR4), and the heavy chain FR residues are positioned about at residues 1-30 (HCFR1), 36-49 (HCFR2), 66-94 (HCFR3), and 103-113 (HCFR4) in the heavy chain residues. The “EU index as in Kabat” refers to the residue numbering of the human IgG1 EU antibody.

Other CDR numbering systems are also used in the art (see, for example, Table A). Chothia and coworkers found that certain sub-portions within Kabat CDRs adopt nearly identical peptide backbone conformations, despite having great diversity at the level of amino acid sequence. (Chothia et al. (1987) J. Mol. Biol. 196: 901-917; and Chothia et al. (1989) Nature 342: 877-883). These sub-portions were designated as L1, L2, and L3 or H1, H2, and H3 where the “L” and the “H” designates the light chain and the heavy chains regions, respectively. These CDRs can be referred to as “Chothia CDRs,” “Chothia numbering,” or “numbered according to Chothia,” and comprise about residues 24-34 (CDR1), 50-56 (CDR2), and 89-97 (CDR3) in the light chain variable domain, and 26-32 (CDR1), 50-56 or 52-56 (CDR2), and 95-102 (CDR3) in the heavy chain variable domain. Mol. Biol. 196:901-917 (1987).

The system described by MacCallum, also referred to as “numbered according to MacCallum,” or “MacCallum numbering” comprises about residues 30-36 (CDR1), 46-55 (CDR2), and 89-96 (CDR3) in the light chain variable domain, and 30-35 (CDR1), 47-58 (CDR2), and 93-101 (CDR3) in the heavy chain variable domain. MacCallum et al. ((1996) J. Mol. Biol. 262(5):732-745).

The system described by AbM, also referred to as “numbering according to AbM,” or “AbM numbering” comprises about residues 24-34 (CDR1), 50-56 (CDR2) and 89-97 (CDR3) in the light chain variable domain, and 26-35 (CDR1), 50-58 (CDR2), and 95-102 (CDR3) in the heavy chain variable domain.

The IMGT (INTERNATIONAL IMMUNOGENETICS INFORMATION SYSTEM) numbering of variable regions can also be used, which is the numbering of the residues in an immunoglobulin variable heavy or light chain according to the methods of the IMGT, as described in Lefranc, M.-P., “The IMGT unique numbering for immunoglobulins, T cell Receptors and Ig-like domains”, The Immunologist, 7, 132-136 (1999), and is expressly incorporated herein in its entirety by reference. As used herein, “IMGT sequence numbering” or “numbered according to IMTG,” refers to numbering of the sequence encoding a variable region according to the IMGT. For the heavy chain variable domain, when numbered according to IMGT, the hypervariable region ranges from amino acid positions 27 to 38 for CDR1, amino acid positions 56 to 65 for CDR2, and amino acid positions 105 to 117 for CDR3. For the light chain variable domain, when numbered according to IMGT, the hypervariable region ranges from amino acid positions 27 to 38 for CDR1, amino acid positions 56 to 65 for CDR2, and amino acid positions 105 to 117 for CDR3.

Other CDR numbering systems and methods are known in the art and can be used herein, such as, for example, those described in A. Sivasubramanian et al. (2017) Broad epitope coverage of a human in vitro antibody library, mAbs, 9:1, 29-42, the contents of which are herein incorporated by reference in its entirety. Combinations of the various CDR numbering systems can also be used in some embodiments.

In some embodiments of the anti-CD277 antibodies described herein, the CDRs recited herein comprise about residues 24-34 (CDR1), 50-56 (CDR2), and 89-97 (CDR3) in the light chain variable domain, and 27-35 (CDR1), 49-60 (CDR2), and 93-102 (CDR3) in the heavy chain variable domain, when numbered according to Chothia numbering. In some embodiments, CDR2 in the light chain variable domain can comprise amino acids 49-56, when numbered according to Chothia numbering. In some embodiments, CDR2 in the heavy chain domain can comprise about residues 50-65, when numbered according to Kabat.

TABLE A
CDR Definitions

| | CDRH1 | CDRH2 | CDRH3 | CDRL1 | CDRL2 | CDRL3 |
|---|---|---|---|---|---|---|
| Kabat | 31-35 | 50-65 | 95-102 | 24-34 | 50-56 | 89-97 |
| Alternative CDRs | 27-35 | 49-60 | 93-102 | 24-34 | 50-56 | 89-97 |
| numbered according | | | | | | |
| to Chothia | | | | | | |
| Chothia | 26-32 | 52-56 or | 95-102 | 24-34 | 50-56 | 89-97 |
| | | 50-56 | | | | |
| MacCallum | 30-35 | 47-58 | 93-101 | 30-36 | 46-55 | 89-96 |
| AbM | 26-35 | 50-58 | 95-102 | 24-34 | 50-56 | 89-97 |
| IMGT | 27-38 | 56-65 | 105-117 | 27-38 | 56-65 | 105-117 |

As used herein, the term “chimeric antibody” means a genetically engineered fusion of parts of an animal antibody, typically a mouse antibody, with parts of a human antibody. Chimeric antibodies are developed to reduce the human anti-animal antibody response elicited by animal antibodies, as they combine the specificity of the animal antibody with the efficient human immune system interaction of a human antibody.

As used herein, the term “chimeric antibody” means a genetically engineered fusion of parts of an animal antibody, typically a mouse antibody, with parts of a human antibody. Chimeric antibodies are developed to reduce a human anti-animal antibody response.

As used herein, the term “co-stimulatory signal” means a signal required for effective activation of a lymphocyte. A non-limiting example of a co-stimulatory signal is a signal generated from engagement of CD28 with one of its cognate ligands CD80 (B7-1) or CD86 (B7-2).

As used herein, the term “cross-reacts” refers to the ability of an antibody of the disclosure to bind to an antigen from a different species. For example, an antibody of the present disclosure which binds human CD277 may also bind another species of CD277. As used herein, cross-reactivity is measured by detecting a specific reactivity with purified antigen in binding assays (e.g., SPR, ELISA) or binding to, or otherwise functionally interacting with, cells physiologically expressing CD277. Methods for determining crossreactivity include standard binding assays as described herein, for example, by BIACORE™ surface plasmon resonance (SPR) analysis using a BIACORE™ 2000 SPR instrument (Biacore AB, Uppsala, Sweden), or flow cytometric techniques.

As used herein, the term “cytotoxic T lymphocyte (CTL) response” refers to an immune response induced by cytotoxic T cells. CTL responses are mediated primarily by CD8⁺ T cells.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In certain embodiments, there is one amino acid difference between a starting polypeptide sequence and the sequence derived there from. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity. In certain embodiments, a polypeptide consists of, consists essentially of, or comprises an amino acid sequence selected from a sequence set forth in Table 2. In certain embodiments, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from a sequence set forth in Table 2. In certain embodiments, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from a sequence set forth in Table 2. In certain embodiments, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from a sequence set forth in Table 2.

In certain embodiments, the antibodies of the disclosure are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like. In certain embodiments, the nucleotide sequence of the invention comprises, consists of, or consists essentially of, a nucleotide sequence selected from a sequence set forth in Table 2. In certain embodiments, a nucleotide sequence includes a nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence selected from a sequence set forth in Table 2. In certain embodiments, a nucleotide sequence includes a contiguous nucleotide sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous nucleotide sequence selected from a sequence set forth in Table 2. In certain embodiments, a nucleotide sequence includes a nucleotide sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous nucleotides of a nucleotide sequence selected from a sequence set forth in Table 2.

It will also be understood by one of ordinary skill in the art that the antibodies suitable for use in the methods disclosed herein can be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The antibodies suitable for use in the methods disclosed herein can comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain embodiments, a string of amino acids can be replaced with a structurally similar string that differs in order and/or composition of side chain family members. Alternatively, in certain embodiments, mutations can be introduced randomly along all or part of a coding sequence, such as by saturation mutagenesis, and the resultant mutants can be incorporated into binding polypeptides of the invention and screened for their ability to bind to the desired target.

As used herein, the term “dimerization” refers to the formation of a macromolecular complex by two, usually non-covalently bound, macromolecules, such as proteins or multimers of proteins. Homodimerization refers to the process of dimerization when the macromolecules (e.g., proteins) are identical in nature. Heterodimerization refers to the process of dimerization when the macromolecules (e.g., proteins) are non-identical in nature.

As used herein, the term “EC₅₀” refers to the concentration of an antibody or an antigen-binding portion thereof, which induces a response, either in an in vitro or an in vivo assay, which is 50% of the maximal response, i.e., halfway between the maximal response and the baseline.

As used herein, the term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “epitope” or “antigenic determinant” refers to a site on an antigen to which an immunoglobulin, antibody, or antigen-binding fragment, specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from CD277 are tested for reactivity with the given anti-CD277 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present disclosure are antibodies that bind to an epitope on CD277 that comprises all or a portion of an epitope recognized by the particular antibodies described herein (e.g., the same or an overlapping region or a region between or spanning the region).

Also encompassed by the present disclosure are antibodies that bind the same epitope and/or antibodies that compete for binding to human CD277 with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as CD277. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope and mass spectrometry combined with hydrogen/deuterium (H/D) exchange which studies the conformation and dynamics of antigen:antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the term “FR” means a framework region.

As used herein, “γδ T cell” refers to a subset of T cells having T cell receptors that express γ and δ chains. Unlike αβ T cells, a γδ T cell recognizes non-common antigens, such as lipid antigens, and plays numerous immune-modulatory roles and immune effector roles by, for example, producing cytokines, such as IFN-γ (see e.g.: Vantourout and Hayday (2013) Nat. Rev. Immunol 13(2): 88-100).

As used herein, the term “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

As used herein, the term “HCDR” means a heavy chain complementarity-determining region.

As used herein, the term “humanized antibody” means an antibody that has variable region framework and constant regions from a human antibody but retains the CDRs of the animal antibody.

As used herein, the term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (See, e.g., Lonberg et al., (1994) Nature 368(6474): 856-859); Lonberg, (1994) Handbook of Experimental Pharmacology 113:49-101; Lonberg & Huszar, (1995) Intern. Rev. Immunol. 13:65-93, and Harding & Lonberg, (1995) Ann. N.Y. Acad. Sci. 764:536-546). However, the term “human antibody” does not include antibodies in which CDR sequences derived from the germline of another mammalian species, such as a mouse, have been grafted onto human framework sequences (i.e. humanized antibodies).

As used herein, the term a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The terms “inducing an immune response” and “enhancing an immune response” are used interchangeably and refer to the stimulation of an immune response (i.e., either passive or adaptive) to a particular antigen. The term “induce” as used with respect to inducing CDC or ADCC refer to the stimulation of particular direct cell killing mechanisms.

As used herein, the terms “inhibits”, “blocks”, or “reduces” (e.g., when referring to inhibition/blocking of the CD277-mediated inhibition of an αβ T cell) are used interchangeably and encompass both partial and complete inhibition/blocking as well as direct and allosteric inhibition/blocking. For example, the inhibition/blocking of CD277 reduces or alters the normal level or type of activity that occurs from CD277 in a given system in the absence of inhibition or blocking. As used herein, “inhibition”, “blocking”, or “reduces” are also intended to include any measurable decrease in biological function and/or activity of a target (e.g. CD277). For example, when an antibody, or an antigen-binding fragment thereof (e.g., an anti-CD277 is in contact with the target as compared to the target not in contact with an antibody, an antigen-binding fragment. In some embodiments, an antibody, or antigen-binding fragment thereof, that targets CD277 inhibits or reduces CD277 function and/or activity in a given system by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an anti-CD277 antibody).

The term “in vivo” refers to processes that occur in a living organism.

As used herein, the terms “induces”, “increases”, “enhances”, or “stimulates” (e.g., when referring to an increase in one or both of αβ T cell and γδ T cell activity or T cell response) are used interchangeably and encompass both increases in activity and de novo activity (e.g., inducing activity from a previously undetectable level). The enhancement of CD277 increases the normal level or type of activity that occurs from CD277 in a given system in the absence of an anti-CD277 antibody or fragment thereof as the enhancer. Enhancement, induction, or stimulation are also intended to include any measurable increase in CD277 activity (or effect on a given cell type) when in contact with an anti-CD277 antibody as compared to CD277 not in contact with an anti-CD277 antibody, e.g., enhances/increases CD277 activity in a given system (CD277-mediated increases in T cell activity) by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, or at least about 1.5-fold, 2-fold, 3-fold, 4-fold, 5-fold, 10-fold, 20-fold, 50-fold, and the like.

The terms “inducing an immune response” and “enhancing an immune response” are used interchangeably and refer the stimulation of an immune response (i.e., either passive or adaptive) to a particular antigen.

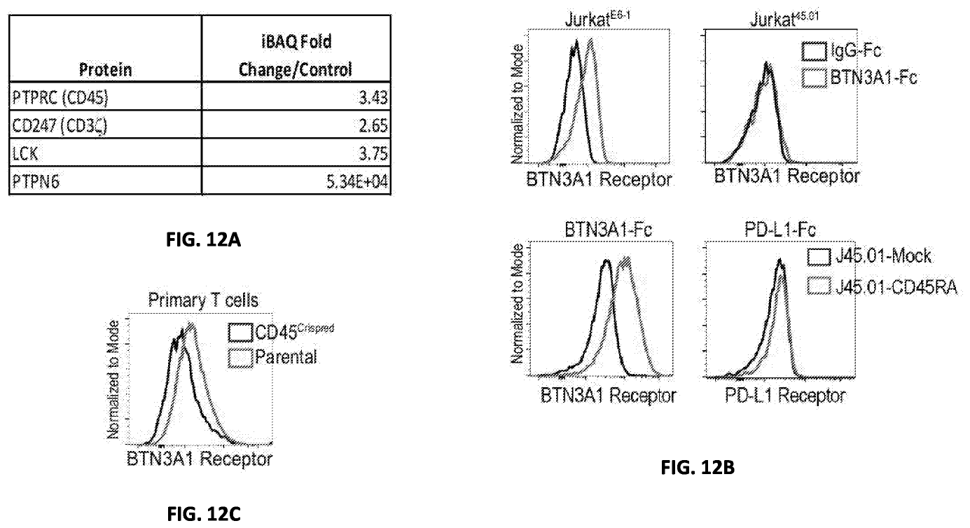

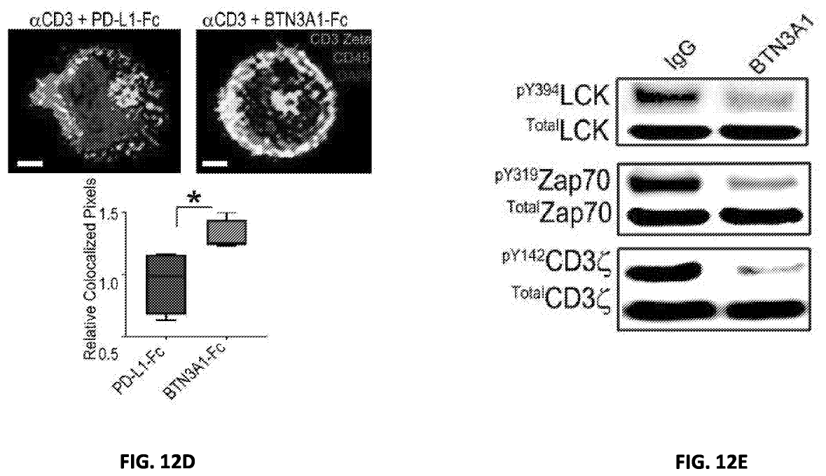

As used herein, the term “interaction” is inclusive of direct and indirect interactions. A non-limiting example of an interaction is the interaction between CD277 and CD45. For example, the interaction between CD277 and CD45 can be a direct interaction or an indirect interaction. Methods to investigate protein-protein interactions are well known in the art (see, for example, Rao et al., International Journal of Proteomics, Vol. 2014, Article ID 147648, 12 pages). Non-limiting examples of methods for investigating protein-protein interactions include co-immunoprecipitation (co-IP), pull-down assays, yeast two-hybrid (Y2H) assays, Far-Western blotting, tandem affinity purification-mass spectroscopy (TAP-MS), protein microarrays, Bio-Layer interferometry (BLI), and Surface Plasmon Resonance (SPR). For example, FIGS. 12A-12E, show results from co-localization experiments used herein to determine interactions between CD277 and CD45/TCR and CD45/CD3.

As used herein, the term “isolated antibody” is intended to refer to an antibody which is substantially free of other antibodies having different antigenic specificities (e.g., an isolated antibody that specifically binds to human CD277 is substantially free of antibodies that specifically bind antigens other than CD277). An isolated antibody that specifically binds to an epitope may, however, have cross-reactivity to other CD277 proteins from different species. However, the antibody continues to display specific binding to human CD277 in a specific binding assay as described herein. In addition, an isolated antibody is typically substantially free of other cellular material and/or chemicals. In some embodiments, a combination of “isolated” antibodies having different CD277 specificities is combined in a well-defined composition.

As used herein, the term “isolated nucleic acid molecule” refers to nucleic acids encoding antibodies or antibody portions (e.g., V_{H}, V_{L}, CDR3) that bind to CD277, is intended to refer to a nucleic acid molecule in which the nucleotide sequences encoding the antibody or antibody portion are free of other nucleotide sequences encoding antibodies or antibody portions that bind antigens other than CD277, which other sequences may naturally flank the nucleic acid in human genomic DNA. For example, a sequence selected from a sequence set forth in Table 2 corresponds to the nucleotide sequences comprising the heavy chain (V_{H}) and light chain (V_{L}) variable regions of anti-CD277 antibody monoclonal antibodies described herein.

As used herein, “isotype” refers to the antibody class (e.g., IgM or IgG1) that is encoded by heavy chain constant region genes. In some embodiments, a human monoclonal antibody of the disclosure is of the IgG1 isotype. In some embodiments, a human monoclonal antibody of the disclosure is of the IgG2 isotype. In some embodiments, a human monoclonal antibody of the disclosure is of the IgG3 isotype. In some embodiments, a human monoclonal antibody of the disclosure is of the IgG4 isotype.

As used herein, the term “isotype switching” refers to the phenomenon by which the class, or isotype, of an antibody changes from one Ig class to one of the other Ig classes.

As used herein the term “KD” or “K_{D}” refers to the equilibrium dissociation constant of a binding reaction between an antibody and an antigen. The value of K_{D} is a numeric representation of the ratio of the antibody off-rate constant (kd) to the antibody on-rate constant (ka). The value of KD is inversely related to the binding affinity of an antibody to an antigen. The smaller the K_{D} value the greater the affinity of the antibody for its antigen. Affinity is the strength of binding of a single molecule to its ligand and is typically measured and reported by the equilibrium dissociation constant (K_{D}), which is used to evaluate and rank order strengths of bimolecular interactions.

As used herein, the term “kd” or “k_{d}” (alternatively “koff” or “k_{off}”) is intended to refer to the off-rate constant for the dissociation of an antibody from an antibody/antigen complex. The value of kd is a numeric representation of the fraction of complexes that decay or dissociate per second, and is expressed in units sec-1.

As used herein, the term “ka” or “kₐ” (alternatively “kon” or “kₒₙ”) is intended to refer to the on-rate constant for the association of an antibody with an antigen. The value of ka is a numeric representation of the number of antibody/antigen complexes formed per second in a 1 molar (1M) solution of antibody and antigen, and is expressed in units M-1 sec-1.

As used herein, the term “LCDR” means a light chain complementarity-determining region.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

As used herein, “MHC molecules” refers to two types of molecules, MHC class I and MHC class II. MHC class I molecules present antigen to specific CD8+ T cells and MHC class II molecules present antigen to specific CD4+ T cells. Antigens delivered exogenously to APCs are processed primarily for association with MHC class II. In contrast, antigens delivered endogenously to APCs are processed primarily for association with MHC class I.

As used herein, the term “monoclonal antibody” refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In some embodiments, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the term “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “patient” includes human and other mammalian subjects that receive either prophylactic or therapeutic treatment.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the term “phosphoantigen” means any non-peptidic antigens that include organic pyrophosphates.

As used herein, the terms “polypeptide,” “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition which reduces the frequency of, or delays the onset of, symptoms of a medical condition in a subject relative to a subject which does not receive the composition.

As used herein, the term “purified” or “isolated” as applied to any of the proteins (antibodies or fragments) described herein refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the term “rearranged” refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

As used herein, the term “receptor clustering” refers to a cellular process that results in grouping or local accumulation of a set of receptors at a particular cellular location, often to induce or amplify a signaling response. Many protein receptors bind cognate ligands and cluster, i.e., form dimers, trimers, oligomers or multimers, upon binding their cognate ligands. Cognate ligand-induced clustering (e.g., dimerization, multimerization) induces signal transduction through the receptor. Accordingly, in some embodiments, the antibodies, or antigen-binding fragments thereof, of the present disclosure can activate a receptor by binding to more than one receptor and induce or stabilize dimerization, trimerization, and/or multimerization with or without cognate ligand binding.

As used herein, the term “recombinant host cell” (or simply “host cell”) is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9):1117-1125), the variable region contains the antigen-binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

As used herein, the term “SEQ ID NO” is synonymous with the term “Sequence ID No.”

As used herein, the terms “specific binding,” “selective binding,” “selectively binds,” and “specifically binds,” refer to an antibody binding to an epitope on a predetermined antigen. The terms also apply to an antagonist binding to a target. Typically, the antibody or antagonist binds with an equilibrium dissociation constant (Kd) of approximately less than 10-6 M, such as approximately less than 10-7, 10-8 M, 10-9 M or 10-10 M or even lower when determined by surface plasmon resonance (SPR) technology in a BIACORE 2000 instrument. For example, when using the recombinant human CD277 extracellular domain as the analyte/predetermined antigen and an anti-CD277 antibody as the ligand, SPR can be used to measure binding of the ligand/antibody to the analyte/predetermined antigen with an affinity that is at least about two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present disclosure, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

As used herein, the term “tumor microenvironment” (alternatively “cancer microenvironment”; abbreviated TME) refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the TME. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells can be the target cell type for a method of use of the invention.

As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof.

As used herein, the term “T cell-mediated response” or “T cell response” refers to any response mediated by T cells, including, but not limited to, effector T cells (e.g., CD8⁺ cells) and helper T cells (e.g., CD4⁺ cells)) and gamma delta T cells (γδ T cells), including Vγ9/Vδ2 T cells. T cell mediated responses include, for example, T cell cytotoxicity, T cell proliferation, T cell cytokine production, and T cell expression patterns. Accordingly, in those aspects and embodiments where enhanced or increased T cell responses are measured, such enhancement or increases encompass both increases in activity and de novo activity (e.g., inducing activity from a previously undetectable level), and can be measured using techniques known in the art for detecting changes in cell surface molecule expression, cytokine production, proliferation, and cytotoxicity, including, but not limited to, flow cytometric-based methods, ELISA-based methods, microscopy-based methods, and combinations thereof.

As used herein, the terms “therapeutically effective amount” or “therapeutically effective dose,” or similar terms used herein are intended to mean an amount of an agent (e.g., an anti-CD277 antibody or an antigen-binding fragment thereof) that will elicit the desired biological or medical response (e.g., an improvement in one or more symptoms of a cancer).

The terms “treat,” “treating,” and “treatment,” as used herein, refer to therapeutic or preventative measures described herein. The methods of “treatment” employ administration to a subject, in need of such treatment, a human antibody of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “unrearranged” or “germline configuration” refers to the configuration wherein the V segment is not recombined so as to be immediately adjacent to a D or J segment.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “VH” means a variable heavy domain.

As used herein, the term “VL” means a variable light domain.

Reference to particular amino acids may be made in respect of common 1-letter or 3-letter codes as commonly understood by persons skilled in the art. Any reference to an “X” amino acid is reference to a variable amino acid. Amino acids can be modified as described herein.

Unless otherwise defined, all technical and scientific terms used herein have the same meaning as commonly understood by one of ordinary skill in the art to which this disclosure pertains. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entireties.

### CD45

As described herein, BTN3A1 (also referred to herein as CD277) suppresses αβ T cells, in part, by preventing the segregation of CD45 from the immunological synapse, which is required for optimal T cell signaling following TCR engagement with cognate MHC. Accordingly, by targeting BTN3A1 using the antigen-binding agents, antibodies and antigen-binding portions thereof described herein, the interaction with CD45 can be inhibited, leading to activation or enhancement of αβ T cell responses.

CD45 (also known as Ly-5 or leukocyte common antigen) is an evolutionary highly conserved receptor protein tyrosine phosphatase exclusively expressed on all nucleated cells of the hematopoietic system. CD45 is a large glycoprotein of 180-220 kDa and constitutes 5-10% of the total glycoprotein on the surface of T- and B-lymphocytes (Rheinlander et al., CD45 in human physiology and clinical medicine, Immunology Letters; Volume 196; p: 22-32 (2018)).

CD45 is characterized by the expression of several isoforms, specific to a certain cell type and the developmental or activation status of the cell. These different isoforms of CD45 are generated by differential splicing of exons 4, 5, and 6, thereby generating the CD45RA, RB and RC isoforms, respectively. These exons encode a sequence of about 200 amino acids close to the extracellular N-terminus of CD45, which contains multiple sites for O-linked glycosylation. Thus, different CD45 isoforms vary in glycosylation patterns and size. The remaining extracellular domain contains a cysteine-rich region and three fibronectin type III repeats, which are heavily N-glycosylated. These complex N-glycans are necessary for CD45 stability and its transport to the cell surface. The membrane-proximal region of CD45 is followed by a single transmembrane region and a long cytoplasmic tail, which harbors a tandem repeat of two tyrosine phosphatase domains, D1 and D2. Only D1 has tyrosine phosphatase activity (PTA) while the D2 domain binds to the cytoskeleton through the linker protein fodrin and acts as a regulator of D1 tyrosine PTA and specificity. CD45 isoform expression varies depending on the stage of T-cell maturation, activation and differentiation. Naïve human T cells express the high molecular weight isoform containing exon 4, CD45RA. After cell activation, the extracellular domain of CD45RA undergoes alternative splicing and is replaced by CD45RO, which is also found on memory T cells.

CD45 is one of the key players in the initiation of T cell receptor signaling by controlling the activation of the Src family protein-tyrosine kinases, Lck and Fyn. When T cells encounter cognate antigen presented on MHC molecules of antigen presenting cells (APCs) they form long-lasting cell conjugates and build an “immunological synapse” or “immune synapse” in the T cell-APC contact zone, which is essential for T-cell activation. CD45 and Lck are initially recruited to the central supramolecular activation cluster (cSMAC) via the TCR. CD45 is then expelled from the cSMAC and clusters in the distal SMAC (dSMAC). Without wishing to be bound or limited by theory, the ‘kinetic-segregation’ model proposes that TCR signaling requires spatial segregation of MHC-bound TCRs from phosphatases. Exclusion of CD45 from the narrow-spaced TCR-MHC interaction zone is believed to result from steric hindrance due to the large size and rigidity of the CD45 extracellular domain. Low or medium local concentrations of CD45 lead to dephosphorylation of the Src family protein-tyrosine kinase Lck (lymphocyte specific kinase) at its C-terminal negative regulatory tyrosine Y505, thereby inducing an opening of the molecule and generating ‘primed’ Lck. In addition, CD45 dephosphorylates and inhibits tyrosine kinases of the Janus kinase (JAK) family, which activate transcription factors of the STAT (signal transducers and activators of transcription) family, crucial regulators of cytokine and chemokine gene expression, thus linking CD45 to cytokine/chemokine responses. The interaction between CD45 and Src kinases is vital for successful antigen receptor signaling in T and B cells. Other CD45 substrates include the CD3ζ and CDR chains and tyrosine kinase Zap 70.

Thus, in some aspects, provided herein are antigen-binding agents, antibodies, or antigen-binding portions thereof that specifically bind to human CD277 and promote CD45 segregation or exclusion from the TCR/MHC immunological synapse, thereby activating or enhancing one or more αβ T cell responses. In some aspects, antigen-binding agents, antibodies, or antigen-binding portions thereof are provided that bind to human CD277 and promote CD45 segregation from CD45 substrates, like CD3ζ, thereby activating or enhancing one or more αβ T cell responses. In some aspects, provided herein are antigen-binding agents, antibodies, or antigen-binding portions thereof that bind human CD277 and promote, increase, or enhance the level of phosphorylation of one or more TCR signaling molecules found at the immunological synapse, such as LCK, Zap70, and CD3ζ, thereby activating or enhancing one or more αβ T cell responses. In some embodiments of such aspects, binding of human CD277 by such antibodies or antigen-binding portions promotes phosphorylation of activating residues of one or more TCR signaling molecules found at the immunological synapse, such as LCKP^{pY394}, Zap70^{pY319}, and CD3ζ^{pY142}, thereby activating or enhancing αβ T cell responses.

### Anti-CD277 Antigen-Binding Agents, Antibodies and Antigen-Binding Fragments Thereof

Provided herein, in some aspects, are antigen-binding agents that specifically bind CD277. Such antigen-binding agents can be a single multifunctional polypeptide, small molecule, or aptamer, or can be a multimeric complex of two or more molecules that are covalently or non-covalently associated with one another. Antigen-binding agents that specifically bind CD277 can, in various aspects and embodiments, comprise one or more antibodies and/or antigen-binding portions thereof. For example, an antigen-binding unit can comprise a variable heavy and/or variable light chain, or complementarity determining regions thereof, of a given antibody to CD277. In some embodiments, the antigen-binding agents that specifically bind CD277 can comprise, in part, scaffold domains, proteins, or portions thereof, e.g., molecules which do not provide target receptor-binding activity, but which can provide a portion or domain of the construct which provides spatial organization, structural support, a means of linking of multiple receptor-binding units, or other desired characteristics, e.g., improved half-life, as described elsewhere herein.

Provided herein, in some aspects, are isolated antibodies, or antigen-binding fragments thereof, which bind to human CD277. CD277 is a member of the butyrophilin subfamily 3 (BTN3), and shares sequence similarities and structural features with certain B7 family members. CD277 is expressed on numerous immune cell types including T cells, NK cells, and antigen-presenting cells such as macrophages and dendritic cells (see for e.g.: Messal et al. (2011) Eur. J. Immunol. 41(12): 3443-54; and Cubillos-Ruiz et al. (2010) Oncotarget 1(5) 329-38). An exemplary amino acid sequence for human CD277 is as follows:

MKMASFLAFLLLNFRVCLLLLQLLMPHSAQFSVLGPSGPILAMVGEDAD
LPCHLFPTMSAETMELKWVSSSLRQVVNVYADGKEVEDRQSAPYRGRTS
ILRDGITAGKAALRIHNVTASDSGKYLCYFQDGDFYEKALVELKVAALG
SDLHVDVKGYKDGGIHLECRSTGWYPQPQIQWSNNKGENIPTVEAPVVA
DGVGLYAVAASVIMRGSSGEGVSCTIRSSLLGLEKTASISIADPFFRSA
QRWIAALAGTLPVLLLLLGGAGYFLWQQQEEKKTQFRKKKREQELREMA
WSTMKQEQSTRVKLLEELRWRSIQYASRGERHSAYNEWKKALFKPADVI
LDP33KTANPILLVSEDQRSVQRAKEPQDLPDNPERFNWHYCVLGCESF
ISGRHYWEVEVGDRKEWHIGVCSKNVQRKGWVKMTPENGFWTMGLTDGN
KYRTLTEPRTNLKLPKPPKKVGVFLDYETGDISFYNAVDGSHIHTFLDV
SFSEALYPVFRILTLEPTALTICPA (SEQ ID NO: 2,
Uniprot ID No. 000481).

In some embodiments, an anti-CD277 antibody or fragment thereof, described herein, activates cytokine production and/or proliferation of γδ T cells. In some embodiments, the antibody or antigen-binding fragment induces or enhances CD277-mediated γδ T cell stimulation in the absence of one or both of: (i) a phosphoantigen (e.g., in the absence of phosphoantigen accumulation, such as phosphoantigen accumulation resulting from treatment of a cell with zoledronate), and (ii) a co-stimulatory signal. In some embodiments, the antibody or antigen-binding fragment reduces CD277-mediated inhibition of γδ T cells in the absence of one or both of: (i) a phosphoantigen (e.g., in the absence of phosphoantigen accumulation, such as phosphoantigen accumulation resulting from treatment of a cell with zoledronate), and (ii) a co-stimulatory signal. Phosphoantigen accumulation within a cell (e.g., a T cell, such as a γδ T cell) may be promoted by a bisphosphonate, such as zoledronate. Thus, in some embodiments, an antibody or antigen-binding fragment thereof described herein can induce or enhance CD277-mediated γδ T cell stimulation in the absence of zoledronate, which by way of promoting accumulation of phosphoantigens in a cell, can itself promote CD277-mediated γδ T cell stimulation. In embodiments, the co-stimulatory signal results from CD3 engagement or CD28 engagement.

In some embodiments, the anti-CD277 antibody or fragment thereof co-stimulates T cells. In various embodiments, the anti-CD277 antibody or fragment thereof co-stimulates T cells together with CD3-TCR, and/or CD28-B7 engagement.

In some embodiments, the isolated antibody or antigen-binding fragment thereof induces or enhances CD277-mediated γδ T cell proliferation. In further embodiments, the isolated antibody or antigen-binding fragment thereof induces or enhances CD277-mediated cytokine production by a γδ T cell. The cytokine production may be IFNγ production.

In some embodiments, the isolated antibody or antigen-binding fragment thereof induces or enhances CD277-mediated αβ T cell proliferation. In further embodiments, the isolated antibody or antigen-binding fragment thereof induces or enhances CD277-mediated cytokine production by a αβ T cell. The cytokine production may be IFNγ production.

In some embodiments, the disclosure features an isolated antibody or antigen-binding fragment thereof that binds to human CD277, wherein the antibody or antigen-binding fragment reduces CD277-mediated inhibition of αβ T cells in the absence of one or both of: (i) a phosphoantigen (e.g., in the absence of phosphoantigen accumulation, such as phosphoantigen accumulation resulting from treatment of a cell with zoledronate), and (ii) a co-stimulatory signal. In some embodiments, the reduction of CD277-mediated inhibition of αβ T cells is CD277-mediated αβ T cell proliferation. In some embodiments, the reduction of CD277-mediated inhibition of αβ T cells is CD277-mediated cytokine production by a αβ T cell. In some embodiments, the cytokine production is IFNγ production.

In some embodiments, the anti-CD277 antibodies described herein comprise heavy chain CDR1, CDR2 and CDR3 sequences set forth in SEQ ID NOs: 7-9, respectively, and light chain CDR1, CDR2 and CDR3 sequences set forth in SEQ ID NOs: 10-12, respectively. In some embodiments, the anti-CD277 antibodies described herein comprise heavy chain CDR1, CDR2 and CDR3 sequences set forth in SEQ ID NOs: 7, 31, and 9, respectively, and light chain CDR1, CDR2 and CDR3 sequences set forth in SEQ ID NOs: 10-12, respectively.

In some embodiments, the anti-CD277 antibodies described herein comprise heavy and light chain variable regions comprising amino acid sequences set forth in SEQ ID NOs: 3 and 4, respectively. In some embodiments, the anti-CD277 antibodies described herein comprise heavy and light chain variable regions comprising amino acid sequences set forth in SEQ ID NOs: 3 and 34, respectively.

In some embodiments, the anti-CD277 antibodies described herein comprise heavy and light chain variable regions, wherein the heavy chain variable region comprises an amino acid sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 3; and wherein the light chain variable region comprises an amino acid sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 4 or SEQ ID NO: 34.

In some embodiments, the anti-CD277 antibodies described herein comprise heavy and light chain variable regions, wherein the heavy chain variable region comprises an amino acid sequence which is at least 90% (e.g., at least 90%, at least 91%, at least 92%, at least 93%, at least 94%, at least 95, at least 96%, at least 97%, at least 98%, or at least 99% identical) identical to SEQ ID NO: 3; and wherein the light chain variable region comprises an amino acid sequence which is at least 90% identical (e.g., at least 90%, at least 91%, at least 92%, at least 93%, at least 94%, at least 95, at least 96%, at least 97%, at least 98%, or at least 99% identical) to SEQ ID NO: 4 or SEQ ID NO: 34. In some such embodiments, the heavy chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 3. In some such embodiments, the light chain variable region comprises an amino acid sequence that differs by 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, 5 amino acids or less, 4 amino acids or less, 3 amino acids or less, 2 amino acids or less, or 1 amino acid from SEQ ID NO: 4 or SEQ ID NO: 34.

In some embodiments, the CDRs of the antibody or antigen-binding portion thereof comprise about residues 24-34 (CDR1), 50-56 (CDR2), and 89-97 (CDR3) in the light chain variable domain of SEQ ID NO: 4, and 27-35 (CDR1), 49-60 (CDR2), and 93-102 (CDR3) in the heavy chain variable domain of SEQ ID NO: 3, when numbered according to Chothia numbering. In some embodiments, CDR2 in the light chain variable domain of SEQ ID NO: 4 or SEQ ID NO: 34 can comprise amino acids 49-56, when numbered according to Chothia numbering. In some embodiments, CDR2 in the heavy chain variable domain of SEQ ID NO: 3 can comprise amino acids 50-65, when numbered according to Kabat.

The disclosure also provides, in some embodiments, an antibody or antigen-binding portion thereof that comprises heavy chain CDRs of the heavy chain variable region of SEQ ID NO: 3, and light chain CDRs of the light chain variable region of SEQ ID NO: 4 or SEQ ID NO: 34, wherein the heavy and light chain CDR residues are numbered according to Kabat.

The disclosure also provides, in some embodiments, an antibody or antigen-binding portion thereof that comprises heavy chain CDRs of the heavy chain variable region of SEQ ID NO: 3, and light chain CDRs of the light chain variable region of SEQ ID NO: 4 or SEQ ID NO: 34, wherein the heavy and light chain CDR residues are numbered according to Chothia.

The disclosure also provides, in some embodiments, an antibody or antigen-binding portion thereof that comprises heavy chain CDRs of the heavy chain variable regions of SEQ ID NO: 3, and light chain CDRs of the light chain variable region of SEQ ID NO: 4 or SEQ ID NO: 34, wherein the heavy and light chain CDR residues are numbered according to MacCallum.

The disclosure also provides, in some embodiments, an antibody or antigen-binding portion thereof that comprises heavy chain CDRs of the heavy chain variable regions of SEQ ID NO: 3, and light chain CDRs of the light chain variable region of SEQ ID NO: 4 or SEQ ID NO: 34, wherein the heavy and light chain CDR residues are numbered according to AbM.

The disclosure also provides, in some embodiments, an antibody or antigen-binding portion thereof that comprises heavy chain CDRs of the heavy chain variable regions of SEQ ID NO: 3, and light chain CDRs of the light chain variable region of SEQ ID NO: 4 or SEQ ID NO: 34, wherein the heavy and light chain CDR residues are numbered according to IMGT.

In some embodiments, the anti-CD277 antibodies described herein comprise a heavy chain comprising the amino acid sequence set forth in SEQ ID NO: 26, and a light chain comprising the amino acid sequence set forth in SEQ ID NO: 27.

In some embodiments, the anti-CD277 antibodies described herein comprise a heavy chain comprising an amino acid sequence at least 90% identical (e.g., at least 90%, at least 91%, at least 92%, at least 93%, at least 94%, at least 95, at least 96%, at least 97%, at least 98%, or at least 99% identical) to SEQ ID NO: 26, and a light chain comprising an amino acid sequence at least 90% identical (e.g., at least 90%, at least 91%, at least 92%, at least 93%, at least 94%, at least 95, at least 96%, at least 97%, at least 98%, or at least 99% identical) to SEQ ID NO: 27.

### Methods of Producing Antibodies

The disclosure also features methods for producing any of the antibodies or antigen-binding fragments thereof described herein. In some embodiments, methods for preparing an antibody described herein can include immunizing a subject (e.g., a non-human mammal) with an appropriate immunogen. Suitable immunogens for generating any of the antibodies described herein are set forth herein. For example, to generate an antibody that binds to CD277, a skilled artisan can immunize a suitable subject (e.g., a nonhuman mammal such as a rat, a mouse, a gerbil, a hamster, a dog, a cat, a pig, a goat, a horse, or a non-human primate) with the extracellular domain of human CD277 (e.g., having SEQ ID NO: 13):

QFSVLGPSGPILAMVGEDADLPCHLFPTMSAETMELKWVSSSLRQVVNV
YADGKEVEDRQSAPYRGRTSILRDGITAGKAALRIHNVTASDSGKYLCY
FQDGDFYEKALVELKVAALGSDLHVDVKGYKDGGIHLECRSTGWYPQPQ
IQWSNNKGENIPTVEAPVVADGVGLYAVAASVIMRGSSGEGVSCTIRSS
LLGLEKTASISIADPFFRSAQRWIAALAG.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a CD277 polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybrid cells are then screened for secretion of the desired antibodies (e.g., an antibody that binds to human CD277 and enhances CD277-mediated stimulation of γδ T cells).

In some embodiments, a skilled artisan can identify an antibody of interest from a non-immune biased library as described in, e.g., U.S. Pat. No. 6,300,064 (to Knappik et al.; Morphosys AG) and Schoonbroodt et al. (2005) Nucleic Acids Res 33 (9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

Methods for identifying antibodies using various phage display methods are known in the art. In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen-binding domains are expressed as a recombinantly fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some embodiments, the phage display antibody libraries can be generated using mRNA collected from B cells from the immunized mammals. For example, a splenic cell sample comprising B cells can be isolated from mice immunized with a CD277 polypeptide as described above. mRNA can be isolated from the cells and converted to cDNA using standard molecular biology techniques. See, e.g., Sambrook et al. (1989) “Molecular Cloning: A Laboratory Manual, 2nd Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., human CD277) using any immunological or biochemical based method known in the art. For example, specific binding of an antibody to CD277, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and noncompetitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody.

In some embodiments, the anti-CD277 antibodies described herein comprise an altered heavy chain constant region that has reduced (or no) effector function relative to its corresponding unaltered constant region. Effector functions involving the constant region of the anti-CD277 antibody may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and proinflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

An altered constant region with altered FcR binding affinity and/or ADCC activity and/or altered CDC activity is a polypeptide which has either an enhanced or diminished FcR binding activity and/or ADCC activity and/or CDC activity compared to the unaltered form of the constant region. An altered constant region which displays increased binding to an FcR binds at least one FcR with greater affinity than the unaltered polypeptide. An altered constant region which displays decreased binding to an FcR binds at least one FcR with lower affinity than the unaltered form of the constant region. Such variants which display decreased binding to an FcR may possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity may exhibit either increased or reduced ADCC and/or CDC activity compared to the unaltered constant region. For example, in some embodiments, the anti-CD277 antibody comprising an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. An anti-CD277 antibody described herein comprising an altered constant region displaying reduced ADCC and/or CDC may exhibit reduced or no ADCC and/or CDC activity.

In some embodiments, an anti-CD277 antibody described herein exhibits reduced or no effector function. In some embodiments, an anti-CD277 antibody described herein comprises a hybrid constant region, or a portion thereof, such as a G2/G4 hybrid constant region (see e.g., Burton et al. (1992) Adv Immun 51:1-18; Canfield et al. (1991) J Exp Med 173:1483-1491; and Mueller et al. (1997) Mol Immunol 34(6):441-452). See above.

In some embodiments, an anti-CD277 antibody may contain an altered constant region exhibiting enhanced or reduced complement dependent cytotoxicity (CDC). Modulated CDC activity may be achieved by introducing one or more amino acid substitutions, insertions, or deletions in an Fc region of the antibody. See, e.g., U.S. Pat. No. 6,194,551. Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See, e.g., Caron et al. (1992) J Exp Med 176:1191-1195 and Shopes (1992) Immunol 148:2918-2922; PCT publication nos. WO 99/51642 and WO 94/29351; Duncan and Winter (1988) Nature 322:738-40; and U.S. Pat. Nos. 5,648,260 and 5,624,821.

Any of the antibodies described herein can be screened and/or tested for their ability to modulate any of the activities or functions ascribed to either CD277, either in vitro or in vivo, using any immunological or biochemical-based methods known in the art.

The antigen-binding agents that specifically bind CD277 described herein can comprise, in part, scaffold domains, proteins, or portions, e.g., molecules which do not provide target receptor-binding activity, but which can provide a portion or domain of the construct which provides spatial organization, structural support, a means of linking of multiple receptor-binding units, or other desired characteristics, e.g., improved half-life. Various scaffold technologies and compositions are known in the art and can be readily linked or conjugated to the antigen-binding units described herein. The scaffold domain, protein, or portion can be derived from an antibody or not derived from an antibody. Such scaffold proteins, and domains thereof, are, generally, obtained through combinatorial chemistry-based adaptation of preexisting antigen-binding proteins.

Non-antibody protein scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. These non-antibody scaffolds and the underlying proteins or peptides on which they are based or from which they have been derived are reviewed by, e.g., Simeon and Chen, Protein Cell 9(1): 3-14 (2018); Vazquez-Lombardi et al., Drug Discovery Today 20: 1271-1283 (2015), and by Binz et al., Nature Biotechnol. 23: 1257-1268 (2005), the contents of each of which are herein incorporated by reference in their entireties. Advantages of using non-antibody scaffolds include increased affinity, target neutralization, and stability. Various non-antibody scaffolds also can overcome some of the limitations of antibody scaffolds, e.g., in terms of tissue penetration, smaller size, and thermostability. Some non-antibody scaffolds can also permit easier construction, not being hindered, for example, by the light chain association issue when bispecific constructs are desired. Methods of constructing constructs on a non-antibody scaffold are known to those of ordinary skill in the art. While not formally on an antibody scaffold, such constructs often include antibody binding domains, whether in the form of single-domain antibodies, scFvs or other antibody binding-domain variants that provide specific target-binding capabilities.

Accordingly, in some embodiments of any of the aspects described herein, an antigen-binding agent can comprise a non-antibody scaffold protein. In some embodiments of any of the aspects described herein, at least one of the agents can comprise a non-antibody scaffold protein. One of skill in the art would appreciate that the scaffold portion of a non-antibody scaffold protein can include, in some embodiments, e.g., an adnectin scaffold or a portion derived from human tenth fibronectin type III domain (10Fn3); an anticalin scaffold derived from human lipocalin (e.g., such as those described in, e.g., WO2015/104406); an avimer scaffold or a protein fragment derived from the A-domain of low density-related protein (LRP) and/or very low density lipoprotein receptor (VLDLR); a fynomer scaffold or portion of the SH3 domain of FYN tyrosine kinase; a kunitz domain scaffold or portion of Kunitz-type protease inhibitors, such as a human trypsin inhibitor, aprotinin (bovine pancreatic trypsin inhibitor), Alzheimer's amyloid precursor protein, and tissue factor pathway inhibitor; a knottin scaffold (cysteine knot miniproteins), such as one based on a trypsin inhibitor from E. elaterium; an affibody scaffold or all or part of the Z domain of S. aureus protein A; a β-Hairpin mimetic scaffold; a Designed ankyrin repeat protein (DARPin) scaffold or artificial protein scaffolds based on ankyrin repeat (AR) proteins; or any scaffold derived or based on human transferrin, human CTLA-4, human crystallin, and human ubiquitin. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al. (2005) TRENDS Biotechnol. 23(10):514-522.

### Recombinant Antigen Binding Agent and Antibody Expression and Purification

The antigen-binding agents, antibodies, or antigen-binding fragments thereof described herein can be produced using a variety of techniques known in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO4 precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999) J Immunol Methods 231(1-2): 147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell, and will be easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Modification of the Antibodies or Antigen-Binding Fragments Thereof

The antibodies or antigen-binding fragments thereof can be modified following their expression and purification. The modifications can be covalent or noncovalent modifications. Such modifications can be introduced into the antibodies or fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments.

In some embodiments, the antibodies or antigen-binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK (SEQ ID NO: 14)), polyhistidine (6-His; HHHHHH (SEQ ID NO: 15), hemagglutinin (HA; YPYDVPDYA (SEQ ID NO: 16)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., 32P, 33P, 14C, 125I, 131I, 35S, and 3H. Suitable fluorescent labels include, without limitation, fluorescein, fluorescein isothiocyanate (FITC), green fluorescent protein (GFP), DYLIGHT™ 488, phycoerythrin (PE), propidium iodide (PI), PerCP, PE-Alexa FLUOR® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins (e.g., an antibody and a heterologous moiety) can be crosslinked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., mmaleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the antibody. Alternatively, the radioactive label can be included as part of a larger molecule (e.g., 125I in meta-[125I]iodophenyl-N-hydroxysuccinimide ([125I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein, or fragment thereof, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the antibodies or antigen-binding fragments thereof can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476 or HESylated (Fresenius Kabi, Germany; see, e.g., Pavisić et al. (2010) Int J Pharm 387(1-2):110-119). The stabilization moiety can improve the stability, or retention of, the antibody (or fragment) by at least about 1.5 (e.g., at about least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### Pharmaceutical Compositions and Formulations

In certain embodiments, the disclosure provides for a pharmaceutical composition comprising an antigen-binding agent that binds CD277 (e.g., an anti-CD277 antibody, or antigen-binding fragment thereof), with a pharmaceutically acceptable diluent, carrier, solubilizer, emulisifier, preservative and/or adjuvant.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and/or rate of in vivo clearance of an antigen-binding agent that binds CD277 (e.g., an anti-CD277 antibody, or antigen-binding fragment thereof).

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an anti-CD277 antibody can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an anti-CD277 antibody can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising an antigen-binding agent that binds CD277 (e.g., an anti-CD277 antibody, or antigen-binding fragment thereof) in a pharmaceutically acceptable vehicle. In certain embodiments, a vehicle for parenteral injection is sterile distilled water in which antigen-binding agent that binds CD277 (e.g., an anti-CD277 antibody, or an antigen-binding fragment thereof) is formulated as a sterile, isotonic solution, and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, an antigen-binding agent that binds CD277 (e.g., an anti-CD277 antibody, or an antigen-binding fragment thereof) can be formulated as a dry powder for inhalation. In certain embodiments, an inhalation solution comprising an antigen-binding agent that binds CD277 (e.g., an anti-CD277 antibody, or an antigen-binding fragment thereof) can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, an antigen-binding agent that binds CD277 (e.g., an anti-CD277 antibody, or an antigen-binding fragment thereof) that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of an antigen-binding agent that binds CD277 (e.g., an anti-CD277 antibody, or an antigen-binding fragment thereof). In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of an antigen-binding agent that binds CD277 (e.g., an anti-CD277 antibody, or an antigen-binding fragment thereof) in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an antigen-binding agent that binds CD277 (e.g., an anti-CD277 antibody, or an antigen-binding fragment thereof) in sustained- or controlled delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an antigen-binding agent that binds CD277 (e.g., an anti-CD277 antibody, or antigen-binding fragment thereof) to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an antigen-binding agent that binds CD277 (e.g., an anti-CD277 antibody, or an antigen-binding fragment thereof) is being used, the route of administration, and the size (body weight, body surface or organ size) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of an antigen-binding agent that binds CD277 (e.g., an anti-CD277 antibody, or an antigen-binding fragment thereof) in the formulation used. In certain embodiments, a clinician will administer the composition until a dosage is reached that achieves the desired effect. In certain embodiments, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising an antigen-binding agent that binds CD277 (e.g., an anti-CD277 antibody, or an antigen-binding fragment thereof) in an ex vivo manner. In such instances, cells, tissues and/or organs that have been removed from the patient are exposed to a pharmaceutical composition comprising an antigen-binding agent that binds CD277 (e.g., an anti-CD277 antibody, or an antigen-binding fragment thereof) after which the cells, tissues and/or organs are subsequently implanted back into the patient.

In certain embodiments, an antigen-binding agent that binds CD277 (e.g., an anti-CD277 antibody, or antigen-binding fragment thereof) can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Kits

A kit can include an antigen-binding agent that binds CD277 (e.g., an anti-CD277 antibody, or antigen-binding fragment thereof) as disclosed herein, and instructions for use. The kits may comprise, in a suitable container, an antigen-binding agent that binds CD277 (e.g., an anti-CD277 antibody, or an antigen-binding fragment thereof), one or more controls, and various buffers, reagents, enzymes and other standard ingredients well known in the art.

The container can include at least one vial, well, test tube, flask, bottle, syringe, or other container means, into which an antigen-binding agent that binds CD277 (e.g., an anti-CD277 antibody, or an antigen-binding fragment thereof) may be placed, and in some instances, suitably aliquoted. Where an additional component is provided, the kit can contain additional containers into which this component may be placed. The kits can also include a means for containing an antigen-binding agent that binds CD277 (e.g., an anti-CD277 antibody, or an antigen-binding fragment thereof) and any other reagent containers in close confinement for commercial sale. Such containers may include injection or blowmolded plastic containers into which the desired vials are retained. Containers and/or kits can include labeling with instructions for use and/or warnings.

### Methods of Use

The compositions of the present invention have numerous in vitro and in vivo utilities. The above-described compositions are useful in, inter alia, methods for treating or preventing a variety of cancers in a subject. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, an antigen-binding agent that binds CD277 (e.g., anti-CD277 antibody, or antigen-binding fragment thereof), is therapeutically delivered to a subject by way of local administration.

A suitable dose of an antigen-binding agent that binds CD277 (e.g., an anti-CD277 antibody, or antigen-binding fragment thereof), described herein, which dose is capable of treating or preventing cancer in a subject, can depend on a variety of factors including, e.g., the age, sex, and weight of a subject to be treated and the particular inhibitor compound used. For example, a different dose of a whole anti-CD277 antibody may be required to treat a subject with cancer as compared to the dose of a CD277-binding Fab′ antibody fragment required to treat the same subject. Other factors affecting the dose administered to the subject include, e.g., the type or severity of the cancer. For example, a subject having metastatic melanoma may require administration of a different dosage of an antigen-binding agent that binds CD277 (e.g., an anti-CD277 antibody, or an antigen-binding fragment thereof) than a subject with glioblastoma. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will also depend upon the judgment of the treating medical practitioner (e.g., doctor or nurse). Suitable dosages are described herein.

A pharmaceutical composition can include a therapeutically effective amount of an antigen-binding agent that binds CD277 (e.g., an anti-CD277 antibody, or antigen-binding fragment thereof), described herein. Such effective amounts can be readily determined by one of ordinary skill in the art based, in part, on the effect of the administered antibody, or the combinatorial effect of the antibody and one or more additional active agents, if more than one agent is used. A therapeutically effective amount of an antigen-binding agent that binds CD277 (e.g., an anti-CD277 antibody, or an antigen-binding fragment thereof)described herein can also vary according to factors such as the disease state, age, sex, and weight of the individual, and the ability of the antibody (and one or more additional active agents) to elicit a desired response in the individual, e.g., reduction in tumor growth. For example, a therapeutically effective amount of an antigen-binding agent that binds CD277 (e.g., an anti-CD277 antibody, or an antigen-binding fragment thereof) can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the antigen-binding agents (e.g., an anti-CD277 antibody, or an antigen-binding fragment thereof)) described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

In some embodiments, the composition contains any of the antigen-binding agents described herein and one or more (e.g., two, three, four, five, six, seven, eight, nine, 10, or 11 or more) additional therapeutic agents such that the composition as a whole is therapeutically effective. For example, a composition can contain an anti-CD277 antibody described herein and an alkylating agent, wherein the antibody and agent are each at a concentration that when combined are therapeutically effective for treating or preventing a cancer (e.g., melanoma) in a subject.

Toxicity and therapeutic efficacy of such compositions can be determined by known pharmaceutical procedures in cell cultures or experimental animals (e.g., animal models of any of the cancers described herein). These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. An antigen-binding agent that binds CD277 (e.g., an anti-CD277 antibody, or an antigen-binding fragment thereof) described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies described herein lies generally within a range of circulating concentrations of the antibodies or antagonists that include the ED₅₀ with little or no toxicity. The dosage may vary within this range depending upon the dosage form employed and the route of administration utilized. For an anti-CD277 antibody described herein, the therapeutically effective dose can be estimated initially from cell culture assays. A dose can be formulated in animal models to achieve a circulating plasma concentration range that includes the IC50 (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

In some embodiments, the methods can be performed in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

As described above, the compositions described herein (e.g., anti-CD277 antibody compositions) can be used to treat a variety of cancers such as but not limited to: Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin' s disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, Kaposi's Sarcoma, skin cancer, stomach cancer, testicular cancer, thyroid cancer, uterine cancer, and cancer of the urinary system. In some embodiments of the methods and uses described herein, the cancer is a solid tumor. In some embodiments of the methods and uses described herein, the cancer is ovarian cancer.

In some embodiments, an antigen-binding agent that binds CD277 (e.g., an anti-CD277 antibody, or antigen-binding fragment thereof) described herein can be administered to a subj ect as a combination therapy with another treatment, e.g., another treatment for a cancer. For example, the combination therapy can include administering to the subject (e.g., a human patient) one or more additional agents that provide a therapeutic benefit to a subject who has, or is at risk of developing, cancer. Chemotherapeutic agents suitable for co-administration with compositions of the present invention include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide. In some embodiments, an antigen-binding agent that binds CD277 (e.g., an anti-CD277 antibody, or an antigen-binding fragment thereof) and the one or more additional active agents are administered at the same time. In other embodiments, the antigen-binding agent that binds CD277 (e.g., an anti-CD277 antibody, or an antigen-binding fragment thereof) is administered first in time and the one or more additional active agents are administered second in time. In some embodiments, the one or more additional active agents are administered first in time and the antigen-binding agent that binds CD277 (e.g., an anti-CD277 antibody, or an antigen-binding fragment thereof) is administered second in time.

In some embodiments, an antigen-binding agent that binds CD277 (e.g., an anti-CD277 antibody, or an antigen-binding fragment thereof), described herein can replace or augment a previously or currently administered therapy. For example, upon treating with an antigen-binding agent that binds CD277 (e.g., an anti-CD277 antibody, or antigen-binding fragment thereof), administration of the one or more additional active agents can cease or diminish, e.g., be administered at lower levels. In some embodiments, administration of the previous therapy can be maintained. In some embodiments, a previous therapy will be maintained until the level of the antigen-binding agent that binds CD277 (e.g., an anti-CD277 antibody, or an antigen-binding fragment thereof) reaches a level sufficient to provide a therapeutic effect. The two therapies can be administered in combination.

Monitoring a subject (e.g., a human patient) for an improvement in a cancer, as defined herein, means evaluating the subject for a change in a disease parameter, e.g., a reduction in tumor growth. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer described herein.

Herein, it has been determined that certain antibodies, such as mAb1, can activate cytokine production and proliferation of T cells (both αβ T cells and γδ T cells) and thereby can be used to overcome the immunosuppressive mechanisms observed in cancer patients.

Accordingly, aspects of the disclosure include methods for treating cancer or delaying progression of cancer or enhancing one or more anti-cancer immune responses in a subject. The methods include: administering to the subject a therapeutically-effective amount of an antigen-binding agent that binds CD277 (e.g., an anti-CD277 antibody, or antigen-binding fragment thereof), wherein the antigen-binding agent induces or enhances CD277-mediated γδ T cell stimulation or reduces CD277-mediated γδ T cell inhibition in the absence of one or both of: (i) a phosphoantigen (e.g., in the absence of phosphoantigen accumulation, such as phosphoantigen accumulation resulting from treatment of a cell with zoledronate), and (ii) a costimulatory signal, to thereby treat the cancer.

In some embodiments, an antigen-binding agent that binds CD277 (e.g., an anti-CD277 antibody, or antigen-binding fragment thereof) described herein induces or enhances CD277-mediated γδ T cell stimulation. In some embodiments, an antigen-binding agent that binds CD277 (e.g., an anti-CD277 antibody, or an antigen-binding fragment thereof) described herein stimulates γδ T cell proliferation and reduces or inhibits αβ T cell activation. In some embodiments, an antigen-binding agent that binds CD277 (e.g., an anti-CD277 antibody, or an antigen-binding fragment thereof) described herein stimulates γδ T cells and inhibits or reduces tumor cell aggregation and metastasis in a subject. The disclosure further provides a method for treating cancer in a subject by stimulating γδ T cells in a subject by administering an antigen-binding agent that binds CD277 (e.g., anti-CD277 antibody, or antigen binding fragment thereof).

In some embodiments, any of the foregoing methods comprise administering to a subject an antigen-binding agent that binds CD277 (e.g., an anti-CD277 antibody, or an antigen binding portion thereof) as described herein.

In some embodiments, γδ T cell stimulation results in γδ T cell activation, proliferation, and/or cytokine production, including, but not limited to, IFNγ production. Increased T cell activation and/or proliferation can be determined by numerous methods known to those persons skilled in the art (see, for e.g. Kruisbeek et al. (2004) Current Protocols in Immunology, 3.12.1-3.12.20). In some embodiments, increased cytokine production involves enhanced IFNγ production, which can be determined by numerous methods including, but not limited to, an ELISA assay. Increased γδ T cell activation, proliferation and/or increased cytokine production from such γδ T cells can be useful biological outputs for targeting a cancer.

In some embodiments, the antigen-binding agent that binds CD277 (e.g., an anti-CD277 antibody, or antigen binding fragment thereof), reduces the CD277-mediated inhibition of αβ T cells. In some embodiments, the reduction of the CD277-mediated inhibition of αβ T cells results in αβ T cell activation, proliferation, and/or cytokine production, including, but not limited to, IFNγ production.

In some embodiments, the γδ T cell stimulation and the reduction of CD277-mediated inhibition of αβ T cells occurs in the absence of one or both of: (i) a phosphoantigen and (ii) a co-stimulatory signal, wherein the co-stimulatory signal results from engagement with CD3 or CD28.

In a particular aspect of the disclosure, an anti-CD277 antibody, such as mAb1, can be used in combination with phosphoantigens. Indeed, phosphoantigens have been shown to activate the cytolytic function of T cells. Without wishing to be bound by theory, it is believed that the use of mAb1 in combination with phosphoantigens can have a synergistic effect. Phosphoantigens have been described in the art (see, for e.g.: WO2007057440 and WO2008059052).

In a particular aspect of the disclosure, anti-CD277 antibodies, such as mAb1, can be used=to treat cancer.

In some embodiments of the methods of treating cancer, or delaying progression of cancer, or enhancing one or more cancer immune responses in a subject in need thereof, the administered antigen-binding agent specifically binds human CD277, and inhibits the interaction of CD277 with CD45, thereby activating or enhancing a αβ T cell response.

In some embodiments of the methods of treating cancer, or delaying progression of cancer, or enhancing one or more cancer immune responses in a subject in need thereof, the administered antigen-binding agent specifically binds human CD277, and inhibits the interaction of CD277 with CD45, thereby disinhibiting the immunosuppressive effect of CD277 toward αβ T cells.

In some embodiments of the methods of treating cancer, or delaying progression of cancer, or enhancing one or more cancer immune responses in a subject in need thereof, the administered antigen-binding agent specifically binds human CD277, and inhibits CD277-mediated association of CD45 with CD3ζ, thereby activating or enhancing one or more αβ T cell responses.

In some embodiments of the methods of treating cancer, or delaying progression of cancer, or enhancing one or more cancer immune responses in a subject in need thereof, the administered antigen-binding agent specifically binds human CD277, and inhibits CD277-mediated association of CD45 with the TCR/MHC immune synapse, thereby activating or enhancing one or more αβ T cell responses.

In some embodiments of the methods of treating cancer, or delaying progression of cancer, or enhancing one or more cancer immune responses in a subject in need thereof, the administered antigen-binding agent specifically binds human CD277, and increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent, or antigen-binding portion thereof, thereby activating or enhancing one or more αβ T cell responses.

Also provided herein, in some aspects, are methods for identifying an antigen-binding agent of interest comprising:

(a) contacting, in the presence of a CD45 protein, a CD277 protein with a test antigen-binding agent; and

(b) identifying the test antigen-binding agent as an antigen-binding agent of interest if the test antigen-binding agent inhibits the interaction between CD45 and CD277.

In some embodiments of these methods and all such methods described herein, one or both of the CD277 protein and the CD45 protein are recombinant proteins. In some embodiments of these methods and all such methods described herein, one or both of the CD277 protein and the CD45 protein is expressed on the surface of cells. In some embodiments of these methods and all such methods described herein, the CD45 protein is expressed on an αβ T cell.

In some aspects, provided herein are methods for identifying an antigen-binding agent of interest comprising:

(a) contacting, in the presence of a CD45 protein, a CD277 protein with a test antigen-binding agent, wherein the CD45 protein is expressed by an αβ T cell; and

(b) identifying the test antigen-binding agent as an antigen-binding agent of interest if the test antigen-binding agent increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent.

In some aspects, provided herein are methods for detecting the immunomodulatory activity of an antigen-binding agent comprising:

(a) detecting the presence, absence, or amount of association of: (i) CD45 with CD3ζ or (ii) CD45 with the TCR/MHC immune synapse on one or more αβ T cells from a subject administered any of the antigen-binding agents described herein, wherein an increase in the association of (i) CD45 with CD3ζ or (ii) CD45 with the TCR/MHC immune synapse on the one or more αβ T cells relative to a control level of association indicates that the antigen-binding agent has immunomodulatory activity.

In some aspects, provided herein are methods for detecting the immunomodulatory activity of an antigen-binding agent comprising:

(a) detecting the presence, absence, level, or amount of phosphorylation of one or more TCR signaling molecules at activating residues selected from the group consisting of LCK^{pY394}, Zap70^{pY319}, and CD3ζ^{pY142} by one or more αβ T cells from a subject administered any of the antigen-binding agents described herein, wherein an increase in the level or amount of phosphorylation of one or more TCR signaling molecules at activating residues selected from the group consisting of LCK^{pY394}, Zap70^{pY319}, and CD3ζ^{pY142} by one or more αβ T cells relative to a control level or amount of phosphorylation, indicates that the antigen-binding agent has immunomodulatory activity.

Aspects of the invention will be illustrated in view of the following figures and examples.

### EXAMPLES

### Example 1

### CD277 Antibody Generation

### CD277 Antigen Preparation

Protein reagent biotinylation was done using the EZ-Link Sulfo-NHS Biotinylation Kit, Thermo Scientific, Cat #21425. The CD277 antigens were concentrated to ˜1 mg/mL and buffer exchanged into PBS before addition of 1:7.5 molar ratio biotinylation reagent (EZ-Link Sulfo-NHS-Biotinylation Kit, Thermo Scientific, Cat #21425.). The mixture was held at 4° C. overnight prior to another buffer exchange to remove free biotin in the solution. Biotinylation was confirmed through Streptavidin sensor binding of the labeled proteins on a ForteBio.

### Library Interrogation and Selection Methodology for Isolation of Anti-CD277 Antibodies Naive Library Selections

Eight naïve human synthetic yeast-based antibody libraries each of ˜10⁹ diversity were designed, generated, and propagated as described previously (see, e.g.,: Xu et al, 2013; WO2009036379; WO2010105256; WO2012009568; Xu et al., Protein Eng Des Sel. 2013 October; 26(10):663-70, all of which are incorporated herein by reference). Eight parallel selections were performed, using the eight naive libraries against biotinylated human CD277 Fc fusion. For the first two rounds of selection, a magnetic bead sorting technique utilizing the Miltenyi MACS system was performed, essentially as described (Siegel et al., J Immunol Methods. 2004 March; 286(1-2):141-53, which is incorporated herein by reference). Briefly, yeast cells (˜10¹⁰ cells/library) were incubated with 10 mL of 10 nM biotinylated human CD277 Fc fusion antigen for 15 min at room temperature in FACS wash buffer PBS with 0.1% BSA. After washing once with 50 mL ice-cold wash buffer, the cell pellet was resuspended in 40 mL wash buffer, and 500 μl Streptavidin MicroBeads (Miltenyi Biotec, Bergisch Gladbach, Germany. Cat #130-048-101) were added to the yeast and incubated for 15 min at 4° C. Next, the yeast were pelleted, resuspended in 5 mL wash buffer, and loaded onto a MACS LS column (Miltenyi Biotec, Bergisch Gladbach, Germany. Cat. #130-042-401). After the 5 mL was loaded, the column was washed three times with 3 ml FACS wash buffer. The column was then removed from the magnetic field, and the yeast were eluted with 5 mL of growth media and then grown overnight.

After the two rounds of MACS, four rounds of sorting were performed using flow cytometry (FACS), which are described in the following three paragraphs.

Selection Strategy Employing 8 Parallel Selections with Fc Antigen

The eight libraries from the MACS selections were taken through four rounds of FACS selections. Approximately 1×10⁸ yeast per library were pelleted, washed three times with wash buffer, and incubated with 10 nM of biotinylated human CD277 Fc fusion antigen for 10 min at room temperature. Yeast were then washed twice and stained with goat anti-human F(ab′)₂ kappa-FITC diluted 1:100 (Southern Biotech, Birmingham, Ala., Cat #2062-02) and either streptavidin-Alexa Fluor 633 (Life Technologies, Grand Island, N.Y., Cat #S21375) diluted 1:500, or Extravidin-phycoerthyrin (Sigma-Aldrich, St Louis, Cat #E4011) diluted 1:50, secondary reagents for 15 min at 4° C. After washing twice with ice-cold wash buffer, the cell pellets were resuspended in 0.4 mL wash buffer and transferred to strainer-capped sort tubes. Sorting was performed using a FACS ARIA sorter (BD Biosciences) and sort gates were determined to select only CD277 binding. The human-CD277 selected populations from the first round of FACS were brought forward into the next round.

The second through fourth round of FACS for the above selected populations involved positive sorts for binders to human and/or cyno CD277 reagents; or negative sorts to decrease polyspecific reagent binders (Xu et al., PEDS. 2013 October; 26(10):663-70). Depending on the amount of polyspecific binding or target binding of a specific selection output, a positive sort followed a negative sort or vice versa, to enrich for a full binding population with limited amount of polyspecific binding. The outputs of these rounds were plated and isolates were picked for sequencing and characterization. One of the isolates characterized was mAb1, detailed herein.

### Affinity Maturation of Clones Identified in Naïve Selections

Heavy chains from the second FACS sorting round output against biotinylated human CD277 Fc fusion were used to prepare light chain diversification libraries. These libraries were used for four additional selection rounds. The first of these selection rounds utilized Miltenyi MACs beads conjugated with 10 nM biotinylated human CD277 Fc fusion as antigen.

Subsequent to the MACs bead selections, three rounds of FACS sorting were performed. The first of these rounds used biotinylated cyno CD277 Fc fusion at 10 nM. The second FACS round for the above involved negative sorts to decrease polyspecific reagent binders as described above. The third and final round of FACS selection was done using biotinylated human monomeric CD277 at 5 nM. Individual colonies from each FACS selection round described above were picked for sequencing characterization.

### IgG and Fab Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over CaptureSelect IgG-CH1 affinity matrix (LifeTechnologies, Cat #1943200250).

### Affinity Measurements of CD2 77 Antibodies

Affinity of the CD277 antibodies was determined by measuring their KD on ForteBio Octet. ForteBio affinity measurements were performed generally as previously described (Estep et al., MAbs. 2013 March-April; 5(2):270-8). Briefly, ForteBio affinity measurements were performed by loading IgGs on-line onto AHQ sensors. Sensors were equilibrated off-line in assay buffer for 30 min and then monitored on-line for 60 seconds for baseline establishment. For avid binding measurement, sensors with loaded IgGs were exposed to 100 nM antigen (human or cyno CD277) for 3 min, afterwards they were transferred to assay buffer for 3 min for off-rate measurement. Monovalent binding measurements were obtained by loading human CD277 Fc fusion on AHQ sensors followed by exposure to 200 nM antibody Fab in solution.

Kinetics data were fit using a 1:1 binding model in the data analysis software provided by ForteBio.

### Octet Red384 Epitope Binning

Epitope binning of the antibodies was performed on a Forte Bio Octet Red3 84 system (Pall Forte Bio Corporation, Menlo Park, Calif.) using a standard sandwich format binning assay. CD277 control antibody IgGs were loaded onto AHQ sensors and unoccupied Fc-binding sites on the sensor were blocked with a non-relevant human IgG1 antibody. The sensors were then exposed to 100 nM target antigen followed by Adimab IgG antibody. Data were processed using ForteBio's Data Analysis Software 7.0. Additional binding by the second antibody after antigen association indicates an unoccupied epitope (non-competitor), while no binding indicates epitope blocking (competitor).

Based on the foregoing methods, a series of anti-CD277 antibodies were generated. Of particular interest, a particular anti-CD277 monoclonal antibody (referred to herein as mAb1) was identified. mAb1 is comprised of the following amino acid sequences:

VH FR1:
(SEQ ID NO: 18)
QVQLVQSGAEVKKPGASVKVSCKASG
VH CDR1:
(SEQ ID NO: 7)
YTFTGYYMH
VH FR2:
(SEQ ID NO: 19)
WVRQAPGQGLEWMG
VH CDR2:
(SEQ ID NO: 8)
WINPNSGGTKYAQKFQG
VH FR3:
(SEQ ID NO: 20)
RVTMTRDTSISTAYMELSRLRSDDTAVYYC
VH CDR3:
(SEQ ID NO: 9)
ARRHSDMIGYYYGMDV
VH FR4:
(SEQ ID NO: 21)
WGQGTTVTVSS
VL FR1:
(SEQ ID NO: 22)
DIQMTQSPSSVSASVGDRVTITC
VL CDR1:
(SEQ ID NO: 10)
RASQGISSWLA
VL FR2:
(SEQ ID NO: 23)
WYQQKPGKAPKLLIY
VL CDR2:
(SEQ ID NO: 11)
AASSLQS
VL FR3:
(SEQ ID NO: 24)
GVPSRFSGSGSGTDFTLTISSLQPEDFATYYC
VL CDR3:
(SEQ ID NO: 12)
QQATDFPPT
VL FR4:
(SEQ ID NO: 25)
FGGGTKVEIK

### Example 2

### Characterization of CD277/BTN3A1 Expression in Dissociated Ovarian Tumors and the Periphery

The materials outlined in Table 1 were used in the Examples as described herein.

TABLE 1
Materials used in Examples

| | Catalogue/Lot | |
|---|---|---|
| Reagent | Number | Source |
| K32/BTN-K32 cells | n/a | Wistar |
| Human PBMCs/Purified | n/a | UPenn Human |
| T cells | | Immunology Core |
| RPMI | 10-040 CM | Corning |
| Penicillin/streptomycin | MT30002C | Corning |
| Fetal calf serum (FCS) | 03-600-511 | Fisher |
| L-Glutamine | MT25005CI | Corning |
| Sodium Pyruvate | 25-000-CI | Corning |
| Anti-human CD3 (OKT3) | BE0001-2 | BioXcell |
| Anti-human CD28 (15E8) | CBL517 | Millipore |
| Recombinant human IL-2 | 200-02 | Peprotech |
| Cell Trace Violet | C34557 | Biolegend |
| Zoledronic Acid | 1724827 | Sigma |
| (Zoledronate) | | |

Human PBMCs and immunopurified CD4+ and CD8+ T cells were obtained from the University of Pennsylvania's human immunology core. These cells were then stained with cell trace violet per the manufacturer's procedure, and were then suspended in RPMI+10% FCS (R10) at a concentration of 1e⁶/ml. Concurrently, K32 and BTN-K32 cells were suspended in R10 at a concentration of 1e⁵ cells/ml, and were then coated with 2 μg/ml anti-CD3 and 1 μg/ml anti-CD28 for 20 minutes at room temperature. K32 and BTN-K32 were then equally divided and were incubated for 20 minutes at room temperature with CD277-specific antibodies (1 μg/ml) provided by Compass. Equal volumes of K32/BTN-K32 cells were mixed with PBMCs/purified CD4+ and CD8+ T cells. Cells were then plated in a 96-well U-bottom plate in a total volume of 200 μl and then incubated at 37° C., 5% CO₂ for 6 days. After this incubation period, cell culture supernatants were collected and subjected to a human IFN-γ ELISA (Biolegend); PBMCs/purified T cells were subjected to analysis by flow cytometry to determine dilution of cell trace violet. In experiments in which zoledronate-pulsed K32/BTN-K32 cells were used, K32 and/or BTN-K32 cells were harvested and plated at 1e⁶/ml in R10 in a 24-well plate. 10 μM zoledronate was then pulsed into the culture and the cells incubated for 24 hours at 37° C., 5% CO₂. These cells were then washed twice and prepped for the proliferation assay as described herein.

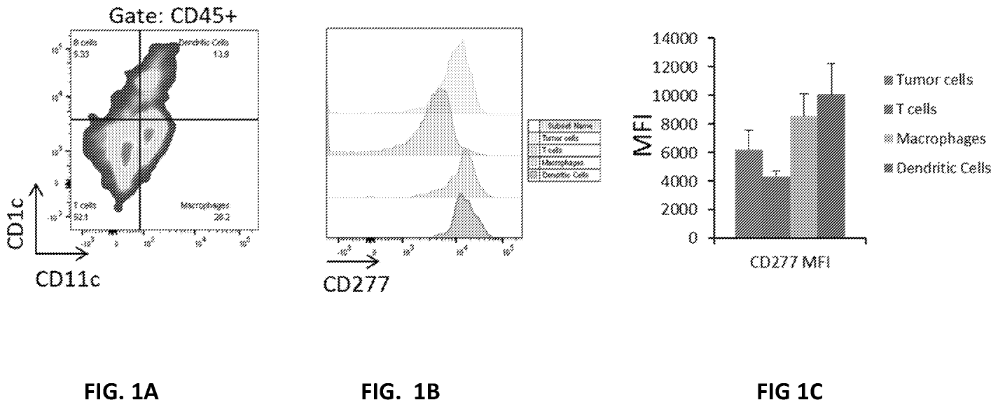

The expression of CD277 was evaluated on infiltrating leukocytes and tumor cells from the dissociated ovarian tumors obtained from four patients by using flow cytometry. The expression of CD277 was also determined on peripheral leukocytes among these patients. As shown in FIGS. 1A-1C, CD277 was highly expressed on multiple lineages of tumor-infiltrating leukocytes; leukocytes observed in the periphery from these tumor-bearing patients express CD277 with the same intensity observed within the TME.

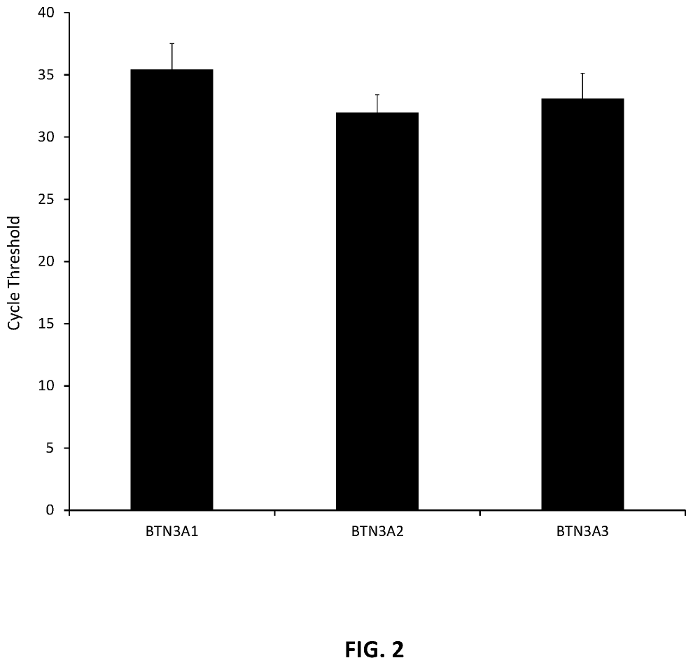

Due to the promiscuity of the CD277 epitope within the butyrophilin subfamily 3, quantitative real-time PCR was employed to quantify levels of BTN3A1, BTN3A2, and BTN3A3 mRNA within ovarian tumors. As shown in FIG. 2, it was observed that the levels of mRNA expression of BTN3A family were similar.

### Example 3

### Characterization of Anti-CD277 Monoclonal Antibodies to Influence αβT Cell Effector Function in the Presence of BTN3A1

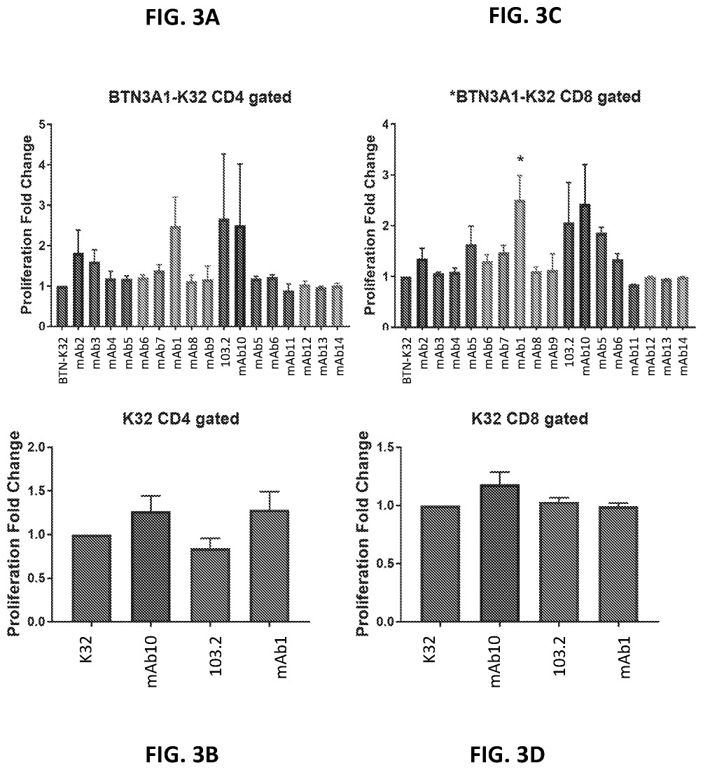

A co-culture system was used to evaluate the suppressive function of K32 cells ectopically expressing human BTN3A1. Total PBMCs or negatively immunopurified CD4+ or CD8+ T cells isolated from healthy donors were cultured in the presence of anti-CD3 (OKT3; 2 μg/ml) and anti-CD28 (15E8; 1 μg/ml) coated K32 cells or K32 cells ectopically expressing human BTN3A1 (BTN-K32) at a 10:1 T cell:K32 ratio, along with BTN3A1-specific antibodies provided by Compass (1 μg/ml). As shown in FIG. 3A, among gated CD4+ T cells within total PBMCs, the application of mAb1 within this in vitro co-culture system improves the proliferative function of gated CD4+ T cells by at least about 1.5 fold (FIG. 3A). Additionally, gated CD8+ T cells also demonstrate enhanced proliferation of at least about 1.5 fold upon the addition of mAb1; this rescue of proliferation achieved statistical significance (FIG. 3C, Right; p<0.05). Importantly, addition of mAb1 did not significantly alter proliferation of gated CD4+ or CD8+ T cells upon stimulation by K32 cells (FIGS. 3B and 3D).

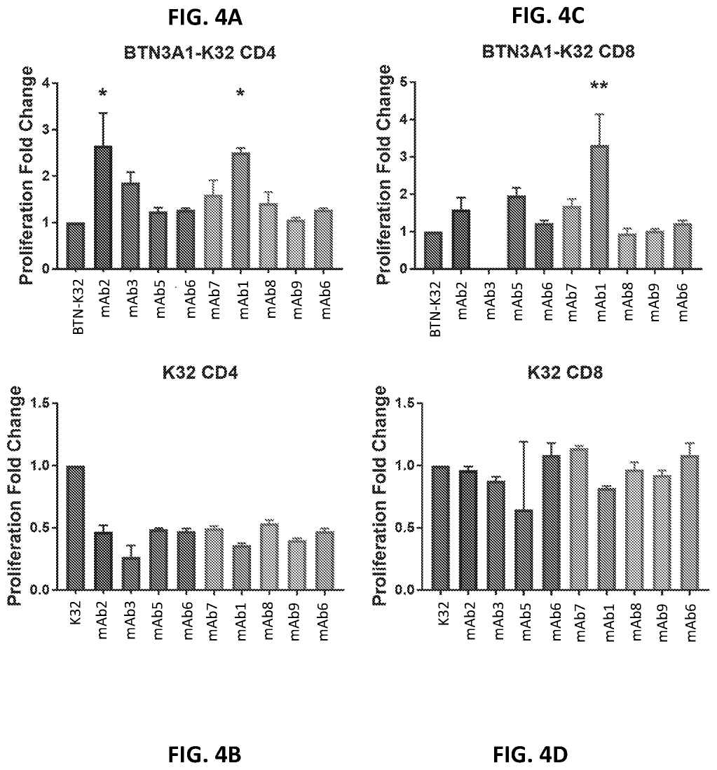

The rescue of T cell proliferation by mAb1 was verified by using negatively immunopurified CD4+ and CD8+ T cells from two healthy patients. FIG. 4A demonstrates that mAb1 significantly improved CD4+ T cell proliferation by at least about 1.5 fold in the presence of BTN3A1. Purified CD8+ T cells stimulated in the presence of BTN3A1 demonstrated a significant at least about 2-fold increase of proliferation upon the addition of mAb1 (FIG. 4C). The addition of these antibodies did not induce non-specific increases in proliferation by T cell when stimulated of K32 cells in the absence of BTN3A1 (FIGS. 3B, 3D and 4B, 4D), however these antibodies may induce a degree proliferative suppression among CD4+ T cells (FIG. 4B).

### Example 4

### Evaluation of the Modulation of the Release of IFN-γ Upon Antibody-Mediated Neutralization of BTN3A1

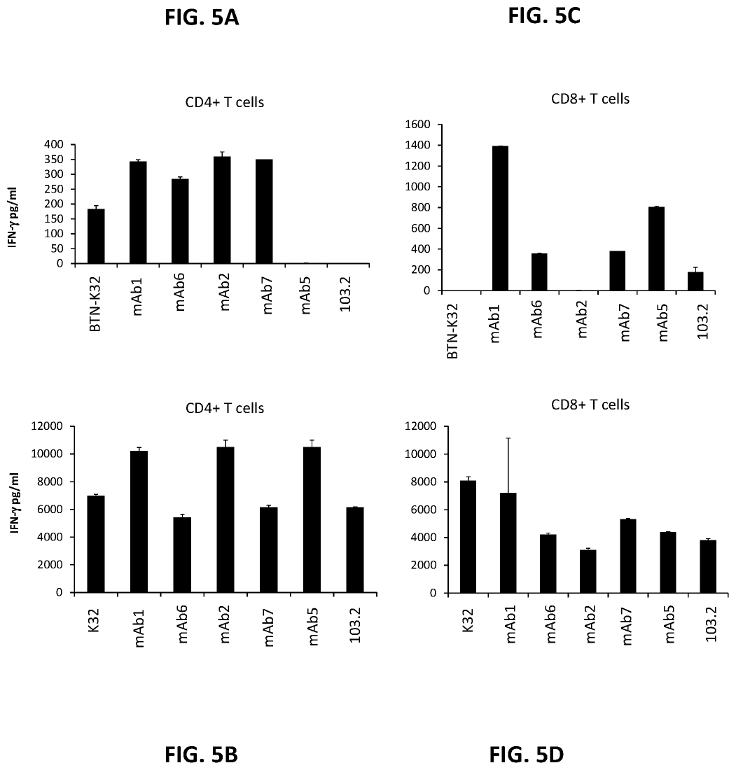

IFN-γ release was used to characterize the improved functionality of immunopurified CD4+ and CD8+ T cells stimulated in the presence of BTN-K32 cells upon the addition of BTN3A1 neutralizing antibodies. As shown in FIG. 5A, the mAb1 was able to enhance the release of IFN-γ by more than at least about 2-fold over baseline for CD4+ T cells. Impressively, mAb1 was shown to improve the release of IFN-γ from CD8+ T cells by about 1300-fold (FIG. 5C). Specificity controls demonstrated some alteration in IFN-γ release upon the addition of antibody for CD4+ T cells, but the differences were less than about 1.5-fold (FIGS. 5B and 5D).

### Example 5

### The Phosphoantigen, Zoledronate, Completely Restores the Proliferative Function of CD4+ and CD8+ T Cells

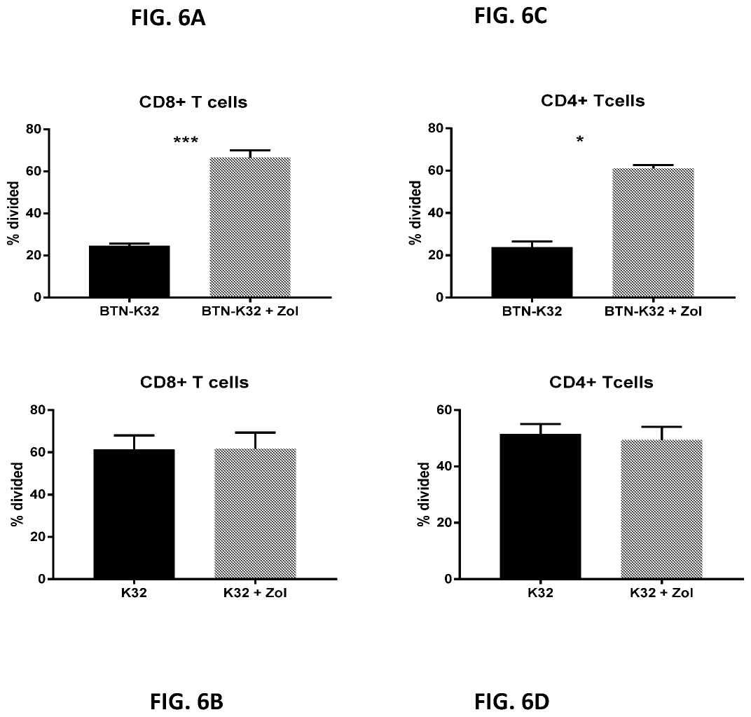

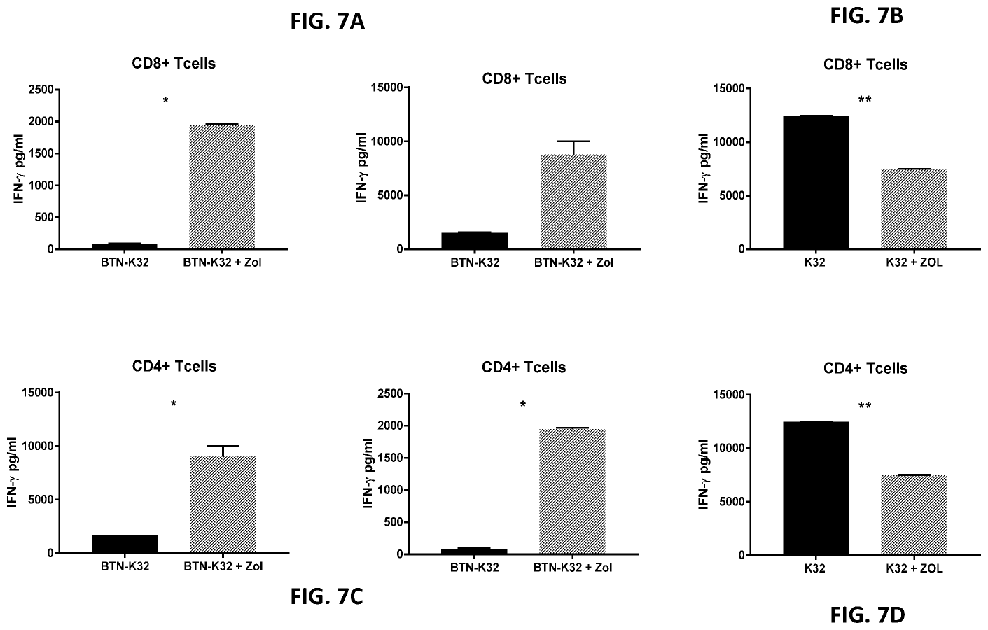

Immunopurified CD4+ and CD8+ T cells were cultured independently in the presence of K32 or BTN-K32 cells that had been treated overnight with 10 μM zoledronate, and then coated with anti-CD3 and anti-CD28 antibodies. As shown in FIGS. 6A-6D, pulsing BTN-K32 cells with zoledronate abrogates the suppressive function of BTN3A1 on both CD4+ and CD8+ T cells (FIGS. 6A and 6C). As expected, no significant differences were observed when stimulating T cells with zoledronate-treated K32 cells (FIGS. 6B and 6D). Furthermore, pre-incubation of BTN-K32 cells with zoledronate dramatically improved the release of IFN-γ in purified CD4+ and CD8+ T cells from two donors (FIGS. 7A, 7B and 7C, 7D) as compared with controls (FIGS. 7E, 7F).

### Example 6

### Zoledronate-Pulsed BTN-K32 Cells Induce the Expansion of γδ T Cells and Their Release of IFN-γ

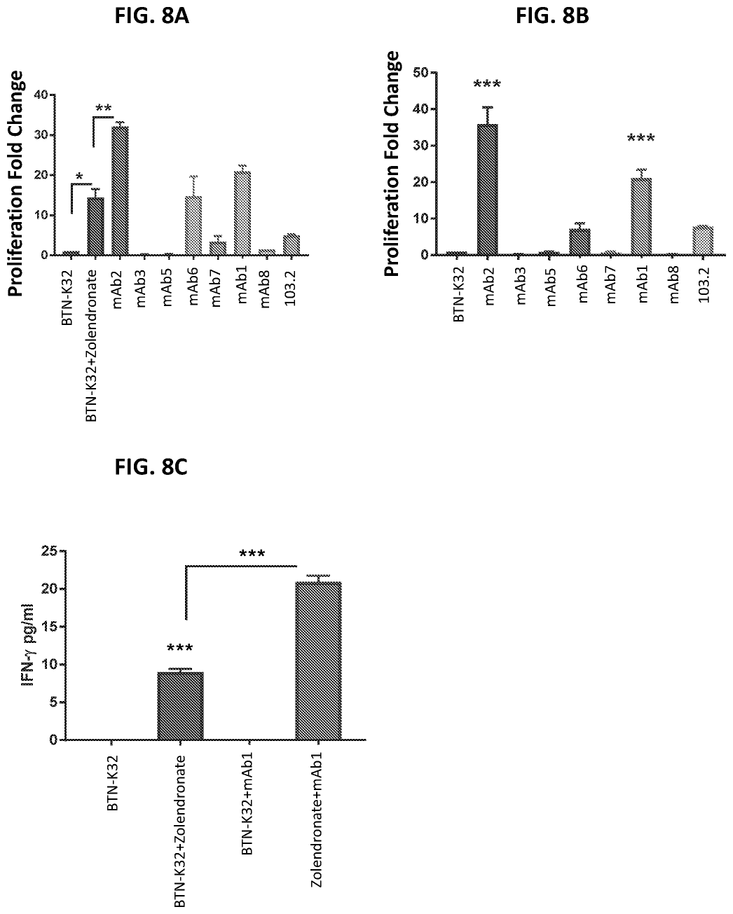

Total PBMCs were cultured in the presence BTN-K32 cells pulsed, or not, with 10 μM zoledronate. Gated γδ T cells demonstrated a significant at least about 15-fold increase in proliferation in the presence of zoledronate-pulsed BTN-K32 cells (FIG. 8). Furthermore, the addition of mAb1 induced significant proliferation of gated γδ T cells stimulated by unpulsed BTN-K32 cells. Immunopurified γδ T cells also demonstrated a significant increase in the release of IFN-γ in the presence of zoledronate-pulsed BTN-K32 cells.

### Example 7

### BTN3A-Specific Antibodies Induce αβ T Cell Proliferation Among Total PBMCs in the Absence of Zoledronate or Supplied Costimulatory Signals 1 and 2

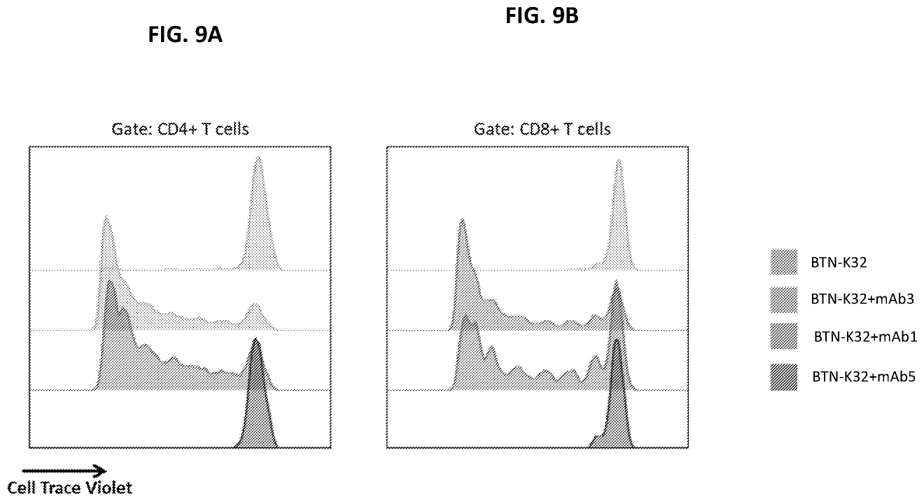

As shown in FIG. 9, BTNA3A-specific antibodies, including mAb1, were shown to induce gated CD4+ and CD8+ T cell proliferation in the absence of zoledronate or supplied signals 1 (CD3) and 2 (CD28). Specifically, PBMCs were cultured in the presence of BTN3-K32 cells in the absence of CD3 or CD28 mAb. Anti-CD277 (1 μg/ml) and recombinant IL-2 (100 U/ml) were added at the initiation of the cultures and proliferation assessed at day 6 by measurement of cell-trace violet signal on gated CD4 and CD8+ T cells.

### Example 8

### In Vivo Anti-Tumor Response with Anti-CD277 Antibody mAb1

To determine whether anti-CD277 antibodies can control tumor growth by enhancing the activity of antigen specific T cells in vivo, a subcutaneous model of xenograft human ovarian cancer that over-expresses CD277 and a model antigen, was utilized.

Specifically, OVCAR3 cells were engineered to over-express CD277 (BTN3A1) and NY-ESO1 by lentiviral infection. Sequences encoding the open-reading frame of BTN3A1 and NYESO-1 were synthesized (IDT) and then ligated into pLVX lentiviral vectors (Takara). After viral packaging, human OVCAR3 tumor cells were transduced to ectopically express both BTN3A1 and NY-ESO1. Over-expression of BTN3A1 and NY-ESO1 compared to parental OVCAR3 was confirmed by western blots (data not shown).

In parallel, sequences encoding an HLA A2-restricted T cell receptor (TCR) that recognizes SLLMWITQC, corresponding to residues 157 to 165 of NY-ESO1 was synthesized (IDT) and ligated into the pBMN-I-GFP retroviral vector, followed by viral packaging. Human donor αβ T cells were successfully transduced to express this specific TCR. Presence of transduced cells was confirmed by flow cytometry (data not shown).

Engineered OVCAR3 tumor cells were then used to challenge immunodeficient NSG mice. Specifically, 5×10⁶ OVCAR3 cells premixed 1:1 with growth factor-reduced matrigel were injected subcutaneously into the axillary flank. Once tumors reached ˜75 mm³, mice received 1×10⁶ NY-ESO1 TCR-transduced αβ T cells intravenously, or mock (empty vector) αβ T cells. Approximately 6 hours after the T cell transfer, administration of antibody mAb1 or IgG control antibody intraperitoneally at 100 μg was initiated and continued every third day until the study end-point. Two separate studies were conducted. Tumor volume was calculated (Length*(Width\hat{ }2)/2) using dial calipers.

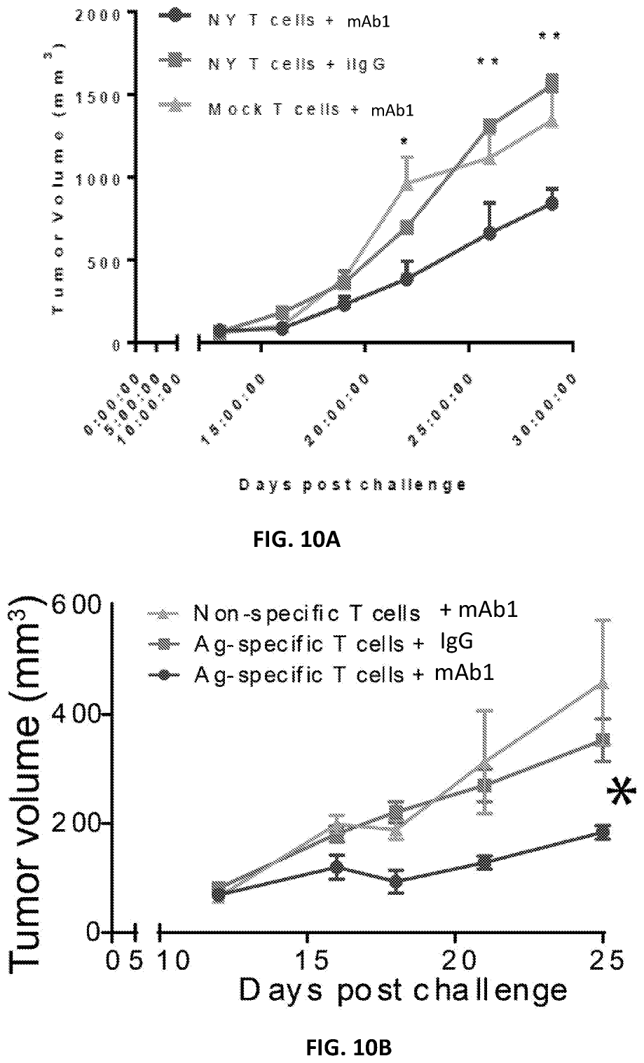

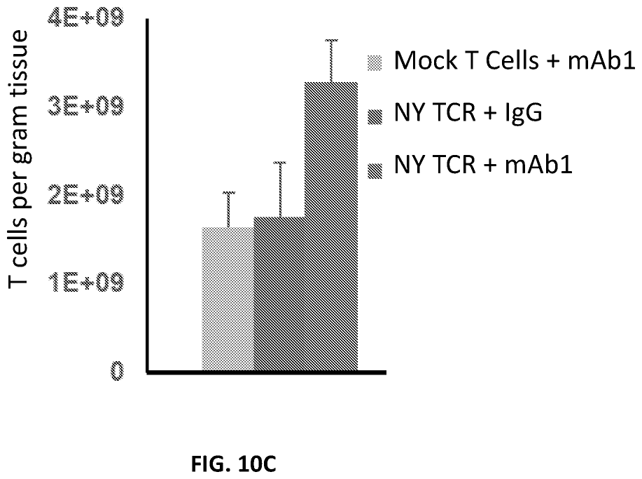

FIGS. 10A and 10B show inhibition of tumor volume growth when antibody mAb1 was administered to mice with NY-ESO-1 TCR-transduced αβ T cells compared to control antibody and compared to mice that received mAb1 with mock αβ T cells. Further, analysis of the tumors at day 30 indicated that antibody mAb1 increased antigen-specific T cell infiltration into BTN3A1+ OVCAR3 tumor microenvironment (FIG. 10C).

Overall, these results indicated that CD277 neutralization enhanced the anti-tumor activity of NY-ESO-1-specific human T cells.

### Example 9

### In Vivo Anti-Tumor Response with Anti-CD277 mAb1 in Adoptive Transfer Model

To determine the effect of anti-CD277 antibodies on in vivo activity of γδ T cells in the tumor microenvironment, OVCAR3 xenografts with an adoptive γδ T cell transfer model was utilized.

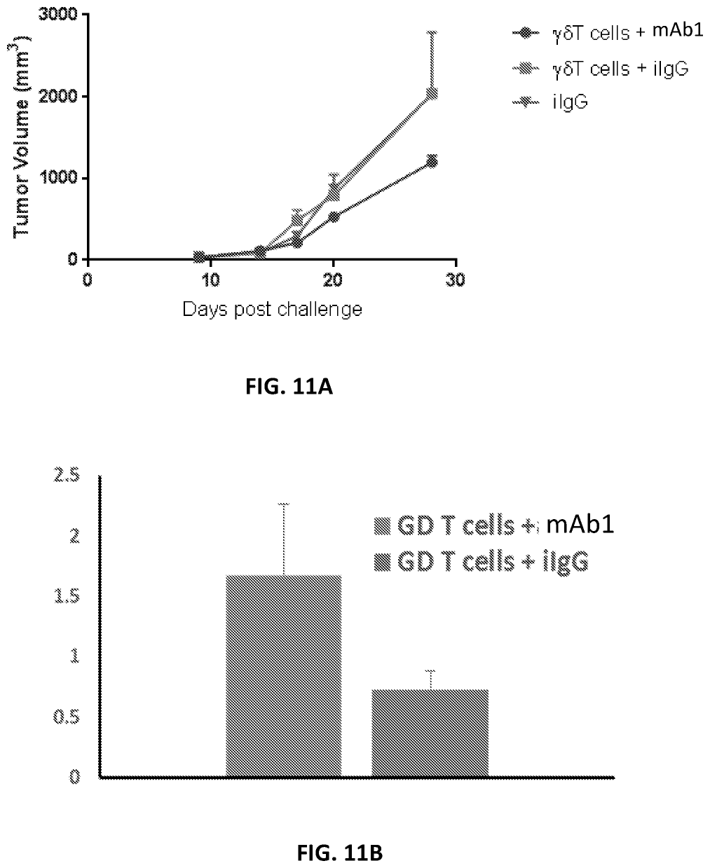

Specifically, NSG mice were challenged with 5×10⁶ BTN3A1+NYESO1+OVCAR3 cells as described above. In addition, Vγ9δ2 γδ T cells from the apheresis of healthy donors were expanded using zolendronate (1 μM) and IL-2 (200 U/ml). After 10 days of expansion, γδ T cells were negatively immunopurified (StemCell Technologies). The purity of Vγ9 cells was greater than 95%. Once tumors reached ˜75mm³, the γδ T cells were serum-starved in HBSS for 4 hours, and then administered intravenously (1×10⁶ cells) to the tumor-bearing mice Approximately 6 hours after T cell transfer, administration of antibody mAb1 or IgG control antibody intraperitoneally at 100 μg was initiated and continued every third day until the study end-point. Tumor volume was calculated (Length*(Width\hat{ }2)/2) using dial calipers. Decreased tumor volume was observed with mAb1 (FIG. 11A). Analysis of the tumors at the end of the study revealed increased accumulation of γδ T cells within ovarian tumors in response to treatment with antibody mAb1 (FIG. 11B).

Overall, these results indicated anti-CD277 antibodies increase the expansion of γδ T cells at tumor beds.

### Example 10

### Materials and Methods

### Genetic Tumor Models and Cell Lines

BTN3A1 (also referred to herein as CD277) knock-in transgenic mice expressing BTN3A1 under the control of the Cd11c promoter were generated at the Fox Chase Cancer Center's Transgenic Mouse Facility (Philadelphia, Pa.) on a C57BL/6 background (A. J. Tesone et al., Satb1 Overexpression Drives Tumor-Promoting Activities in Cancer-Associated Dendritic Cells. Cell Rep 14, 1774-1786 (2016)). Briefly, a construct encoding murine Cd11c was assembled in-frame with BTN3A1 flanked by a rabbit β-globin cassette in a pUC57-Brick plasmid (Genscript; Piscataway, N.J.). Pronuclear injections of the released construct were performed into zygotes from C57BL/6 donors. A single BTN3A1⁺ male was then bred with C57BL/6 females to establish a heterozygous BTN^{TG} colony. NOD.Cg-Prkdc^{scid} Il2rg^{tm1Wjl}/SzJ (NSG) mice were acquired from The Jackson Lab. Wild-type C57BL/6 mice used for bone marrow reconstitution and in vivo tumor challenges were purchased from The Jackson Lab. All animals were maintained and used in accordance with the Institutional Care and Use Guidelines of Moffitt Cancer Center. ID8 cells (K. F. Roby et al., Development of a syngeneic mouse model for events related to ovarian cancer. Carcinogenesis 21, 585-591 (2000)) were provided by K. Roby (Department of Anatomy and Cell Biology, University of Kansas, Kansas City, Kans.) and were retrovirally transduced to express Defb29 and Vegf-a(33). OVCAR3 cells were purchased from ATCC (Manassas, Va.) and were retrovirally transduced to express NY-ESO-1 (OVCAR3^{NY}). OVCAR3^{NY} flank tumors were established by admixing 2e⁶ cells with growth factor-reduced Matrigel (BID Biosciences) at a 1:1 ratio and injecting into the lateral flank. Intraperitoneal ID8-Vegf-Defb29 tumors were initiated by intraperitoneal injection of 5×10⁶ cells. Tumor volume was calculated as: 0.5×(L×W²), where L is length and W is width.

### Constructs

The BTN3A1-Fc fusion protein was purchased from R&D systems: Cat #8539-BT-050. A C-terminus CD3ζ-EGFP fusion protein was generated by digesting pBMN-I-GFP (Addgene) with EcoRI and NcoI to remove the IRES (pBMN^{GFP-Fusion}). CD3ζ with a C-terminus glycine-serine spacer and flanked by 5′ EcoRI and 3′ NcoI restriction sites was then ligated into pBMN^{GFP-Fusion}. A sequence corresponding to an HLA-A2-restricted TCR that recognizes SLLMWITQC (SEQ ID NO: 28), corresponding to residues 157 to 165 of NY-ESO-1 (P. F. Robbins et al., Tumor regression in patients with metastatic synovial cell sarcoma and melanoma using genetically engineered lymphocytes reactive with NY-ESO-1. J Clin Oncol 29, 917-924 (2011)) and publicly available on the worldwide web at google.com/patents/U.S. Pat. No. 8,143,376, was purchased from IDT (Coralville, Iowa) and ligated into pBMN-I-GFP. The ORF of PTPRC (NM_002838.5) was purchased from Genscript (Piscataway, N.J.), and CD45RA was produced by deleting exons 5 and 6. CD45RA was ligated into pLVX-EF1a-IRES-ZsGreen (Takara Bio USA; Mountain View, Calif.). The ORF of CTAG1B (NYESO1) was purchased from IDT and ligated into pLVX-EF1α-IRES-mCherry. The sequence integrity and accuracy of all constructs was assessed and confirmed.

### In Vitro T Cell Activation

1×10⁶ immunopurified αβ T cells were activated in the presence of plate-bound OKT3 (10 μg/ml) and BTN3A1-Fc or IgG-Fc (10 μg/ml) for 1 minute at 37° C. The reaction was then stopped with ice cold PBS, cell pellets were then collected and subjected to immunoblotting.

### Immunoblotting

Protein lysates from frozen ovarian and breast tumor chunks were generated using RIPA buffer (Thermo) after being pulverized in liquid nitrogen. T cell lysates were generated by lysing frozen cell pellets using RIPA buffer. Lysate protein concentrations were quantified using Pierce BCA Protein Assay Kit (#23225) using standardized protocol. Western blots of each total protein lysate were run using XCell II Blot Module (#EI9051) using standard techniques. For ovarian tumor samples PVDF membranes were blotted with primary antibodies, anti-BTN3A1 (Sigma-Aldrich; HPA012565) at 1:500 and β-actin (ThermoFisher; PA1-183) at 1:1000, simultaneously at 4° C. overnight. For T cell samples PVDF membranes were blotted with primary antibodies pY394 LCK (Invitrogen; PA5-37628), total LCK (V49; Cell Signaling), pY319 Zap70 (65E4; Cell Signaling), total Zap70 (99F2; Cell Signaling), pY142 CD3ζ (68235; Abcam), and total CD3ζ (ab226263; Abcam). All antibodies were blotted at 1:1000 at 4° C. overnight. Anti-rabbit HRP (CST; 7074S), was blotted at 1:5000 at room temperature for one 1 h for all membranes. Membranes were imaged using Bio-Rad ChemiDoc Imaging System.

Virus Production and Transduction of T cells

Retroviral particles were generated by transfecting Amphotropic Phoenix cells with pBMN-I-GFP or pBMN^{GFP-Fusion} and T cells were prepared for adoptive cell transfer or in vitro assays as previously described (A. Perales-Puchalt et al., Follicle-Stimulating Hormone Receptor Is Expressed by Most Ovarian Cancer Subtypes and Is a Safe and Effective Immunotherapeutic Target. Clin Cancer Res 23, 441-453 (2017)). Lentiviral particles were generated by transfecting HEK 293T/17 (ATCC) with pLVX-EF1a-IRES-ZsGreen or pLVX-EF1a-IRES-mCherry. 1e5 OVCAR3 cells or Jurkat cells (clone J45.01) were seeded into 6 well culture plates, and transduced with the lentivirus construct by spinfection with 4 μg/ml polybrene (Millipore). 72 hr following transduction, Jurkat J45.01 cells and OVCAR3 cells were sorted for GFP+ and mCherry+ expressing cells, respectively, and passaged into 75 mm flasks. Protein expression was confirmed by FACS and immunoblotting.

### T Cell Proliferation Assay

Human T cell proliferation assays were performed utilizing K562 cells expressing human CD32 (K32 cells), and K562 cells expressing human CD32 and BTN3A1 (BTN-K32 cells), that were generated as described (J. R. Cubillos-Ruiz et al., CD277 is a Negative Co-stimulatory Molecule Universally Expressed by Ovarian Cancer Microenvironmental Cells. Oncotarget 1, 329-328 (2010); M. V. Maus et al., Ex vivo expansion of polyclonal and antigen-specific cytotoxic T lymphocytes by artificial APCs expressing ligands for the T-cell receptor, CD28 and 4-1BB. Nat Biotechnol 20, 143-148 (2002)). These cells were γ-irradiated (100 Gy) and, in some experiments, incubated with anti-CD277 or control antibodies at room temperature for 10 minutes. In some experiments, K32 and BTN-K32 cells were incubated with 1 μM zolendronate (Sigma) overnight at 37° C., and then γ-irradiated. The cells were washed twice with PBS and then loaded with OKT3 (0.5-1 μg/mL; BioXCell) without or with anti-CD28 (100 ng/mL, 15E8; EMD Millipore) antibodies at room temperature for 10 minutes. Immunopurified CD4, CD8, or γδ T cells, were labeled with CELL TRACE VIOLET (Invitrogen) according to the manufacturer's instructions and cocultured with loaded K32 and BTN-K32 cells at a 10:1 T cell:K32 cell ratio. The frequency of proliferated T cells was determined 6 days later by FACS and analyzed using FlowJo software.

### ELISpot

For interferon-γ ELISPOT (eBioscience), 2 million bone marrow-derived dendritic cells (BMDC) were primed with 200,000 ID8-Defb29/Vegf-a overnight. The next day 10,000 T cells derived from the peritoneal wash of mice bearing ID8-Defb29/Vegf-a at day 25 were plated with 1,000 BMDC and the number of spots was measured 72 hours later on an ELISPOT reader using Immunospot software (CTL).

### Flow Cytometry

A BD LSRII flow cytometer or BD FACSARIA cell sorter (BD Biosciences) were used for flow cytometry. Anti-human antibodies were directly fluorochrome conjugated. Specifically, anti-CD3e (OKT3), CD45 (30-F11), CD277 (BT3.1), CD1c (L161), CD11c (3.9), HLA-DR (L243), EpCaM (9C4), Vγ9 (B3), antibodies were used for flow cytometry (all from Biolegend or Tonbo Biosciences). Cell Trace Violet staining was done as directed by the manufacturer (Invitrogen). Live/dead exclusion was performed with Zombie Yellow or 7-AAD (Biolegend).

### Immunohistochemistry

Tissue microarrays from paraffin embedded ovarian tumors were subjected to antigen retrieval and deparaffinized. Slides were then blocked and incubated overnight at 4° C. with anti-BTN3A1 (Atlas; HPA012565) antibody. The following day, slides were washed and then incubated with an HRP-labeled goat anti-rabbit antibody and completion of immunohistochemical procedures, according to the manufacturer's instructions (Biovision). Quantitative acquisition was performed by an automated slide Scanner (Aperio-Leica). Analyses were done using Definiens Tissue Studio version 4.7 software.

### Immunoprecipitation and Proteomics Analysis

Negatively immunopurified T cells were activated in the presence of CD3/CD28 DYNABEADS (Invitrogen) for 24 hrs. T cells were then washed extensively and then incubated on ice with 10 μg/ml BTN3A1-Fc, or irrelevant IgG-Fc, for 1 hour. To induce crosslinking, DTSSP (ThermoFisher) was added at a final concentration of 2 mM and incubated for 30 minutes at room temperature. The reaction was stopped using Tris, pH 7.5, at a final concentration of 20 mM. The cells were then washed extensively in PBS and resuspended in lysis buffer (50 mM Tris-HCl pH8.0, 1 mM EDTA, 1% NP-40, 150 mM NaCl, and protease inhibitors), rotated at 4° C. for 30 min, and cleared by centrifugation at 14,000 g for 10 minutes. Protein lysates were incubated for 2 hours at 4° C. with ProteinG DYNABEADS (ThermoFisher). Beads were washed in PBS with 0.02% Tween 20 three times. Proteins were eluted with LDS sample buffer supplemented with DTT by heating at 70° C. for 10 minutes. Eluted fractions from BTN3A1-Fc and control were run into an SDS-gel for 0.5 cm, and the entire gel lanes were excised and digested with trypsin. Liquid chromatography tandem mass spectrometry (LC-MS/MS) analysis of tryptic digests was performed by the Wistar Proteomics and Metabolomics Facility using a Q Exactive HF mass spectrometer (Thermo Scientific). MS/MS spectra generated from the LC-MS/MS runs were searched using full tryptic specificity against the UniProt human database (www.uniprot.org; Oct. 1, 2017) using the MaxQuant 1.6.0.16 program. Protein quantitation was based on intensity and MS/MS count was determined from unique and razor peptides (J. Cox, M. Mann, MaxQuant enables high peptide identification rates, individualized p.p.b.-range mass accuracies and proteome-wide protein quantification. Nat Biotechnol 26, 1367-1372 (2008)). False discovery rates for protein and peptide were set at 1%. Protein tables were filtered to remove reverse database entries, common contaminants, and proteins identified by a single peptide.

### Confocal Microscopy

Glass poly-L-lysine treated cover slips were coated with OKT3 (10 μg/ml) and BTN3A1-Fc (10 μg/ml) or PD-L1-Fc (10 μg/ml) in PBS overnight at 4° C. 1e6 negatively immunopurified human T cells, previously transduced to express a CD3ζ-EGFP fusion protein and incubated with an α-CD45 antibody (Abcam; MEM-28) and α-mouse AF594 Fab (Cell Signaling), were exposed to the coated coverslips for 3 min at 37° C. and were then fixed in PBS containing 4% paraformaldehyde for 30 min at 37° C. Coverslips were then gently washed 2× with PBS and mounted to glass slides using antifade mountant with DAPI (Cell Signaling). Glass slides were then observed with a Leica TCS SP8 AOBS laser scanning confocal microscope through a 63X/1.4NA oil immersion Plan Apochromat objective. Laser lines at 405 nm, 488 nm and 594 nm were used to excite the DAPI, EGFP (CD3z-EGFP fusion), and Alexa Fluor 594 (CD45) fluorophores respectively. Images were captured in 0.5 uM increments totaling 10 sections at 200 Hz scan speed with photomultiplier detectors using LAS X software version 3.1.5. Confocal images were analyzed using the Definiens Tissue Studio IF v4.4.2 (Definiens AG, Munich, Germany) software suite. Cells were segmented by the nuclear stain, DAPI (blue) and the Alexa Fluor 597 channel was used as membrane marker for cell simulation. The image was analyzed as an 8 bit image and intensity of red and green channel was measured from 0-255 grayscale fluorescent units. Co-localization analysis was measured by the Alexa Fluor 594 and EGFP channel overlap by pixel count and positive co-localized cell counts.

### CRISPR

crRNA targeting PTPRC 5′UUACCACAUGUUGGCUUAGA3′ (SEQ ID NO: 29) or 5′AGUACAUGAAUUAUGAGAUA3′ (SEQ ID NO: 30) (IDT) were reconstituted to 100 in Nuclease-Free Duplex Buffer (IDT). crRNAs were then mixed at equimolar concentrations with ALT-R® CRISPR-Cas9 tracrRNA, ATTO™ 550 (IDT) in a sterile PCR tube. crRNA:tracrRNA duplexes were annealed by heating at 95° C. for 5 min in PCR thermocycler, then slowly cooled to room temperature. 9 μl of crRNA-tracrRNA duplexes were mixed with 6 μl (180 pmol) of TrueCut Cas9 Protein v2 (Invitrogen), followed by incubation at room temperature for 10 min to form Cas9 RNPs. 2e6 purified CD3+ T cells were then resuspended in 100 μl buffer T of the Neon Tranfection System (ThermoFisher). 15 μl of the Cas9 RNPs were added to the resuspended cells and electroporation was performed at 1400V, 10 ms, 3 cycles. Loss of CD45 from the T cell surface was confirmed by FACs 3 days later.

### Statistical Analysis

All statistical assays were performed using GraphPad Prism 5.0 software. Mann-Whitney test were used for calculating differences between means of experimental groups. P-values less than 0.05 were considered significant.

### Example 11

### BTN3A1 Suppresses αβ T Cells by Preventing the Segregation of CD45 from the Immune Synapse

To determine the mechanism by which BTN3A1 (also referred to herein as CD277) inhibits αβ T cell activity, the binding of BTN3A1 to potential immunosuppressive receptors was ruled out by comparing the binding of recombinant NKG2A, GPR174, NRP1, NRP2, PD-1, CTLA4, TIM-3 and BTLA Fc proteins to MHC-I⁻CD32⁺ K562 artificial Antigen-Presenting Cells with BTN3A1 retrovirally expressed on the surface of the cell (BTN-K32 aAPCs) vs. mock-transduced aAPCs. Binding of BTN3A1 to these immunosuppressive receptors was not observed. BTN3A1-Fc proteins were then used to co-immunoprecipitate (co-IP) the binding partner(s) of BTN3A1 on OKT3-activated primary αβ T cells from three independent pull downs from three different donors. Surprisingly, liquid chromatography tandem mass spectrometry (LC-MS/MS) showed that BTN3A1 co-immunoprecipitated with a complex that included only two proteins with an extracellular domain in three independent experiments: CD3ζ and the phosphatase CD45, in addition to the intracellular TCR signaling protein LCK and the phosphatase PTPN6 (SHP-1) (FIG. 12A). In contrast, BTN3A1 did not reproducibly co-IP with other heavily glycosylated and abundant surface molecules, including CD44, CD5, or CD2. Further supporting the specificity of the binding of BTN3A1 to CD45, BTN3A1-Fc proteins bound to Jurkat cells expressing CD45 (clone E6-1), but not to their CD45 negative counterparts (clone J45.01) (FIG. 12B, upper panels). More importantly, CRISPR ablation of CD45 in primary αβ T cells eliminated the binding of BTN3A1 observed in parental (CD45⁺) lymphocytes (FIG. 12C), and, binding of BTN3A1-Fc to the surface of J45.01 cells was reestablished upon forced expression of CD45RA in these cells (FIG. 12B, lower left panel). Binding of control PD-L1-Fc protein to the surface of J45.01 was not observed upon forced expression of CD45RA in these cells (FIG. 12B, lower right panel). Accordingly, expression of BTN3A1 on aAPCs had no effect in CD45-CRISPRed αβ T cells (both CD4⁺ and CD8⁺), while proliferative responses in non-ablated (CD45⁺) T cells in the same cultures were effectively suppressed.

To determine whether BTN3A1 disrupts TCR triggering by preventing the segregation of CD45 from the immune synapse, a CD3ζ-GFP fusion protein was generated and the degree of CD3ζ:CD45 co-localization was assessed in the presence of BTN3A1-Fc or PD-L1-Fc control after crosslinking the TCR in the presence of plate bound OKT3 and BTN3A1-Fc or control IgG-Fc proteins at 10 mg/ml for three minutes. TCR activation in the presence of PD-L1 occurred in the presence of CD45 segregation (FIG. 12D). In contrast, the presence of BTN3A1 impeded the segregation of CD45 from CD3ζ in multiple independent experiments with different donors, further supporting that BTN3A1 abrogates αβ T cell responses by effectively dismantling the immune synapse (FIG. 12D). Consequently, TCR activation in the presence of BTN3A1 dramatically blunted the phosphorylation of TCR signaling molecules proximally at activating residues in LCK^{pY394}, Zap70^{pY319} and CD3ζ^{pY142} (FIG. 12E). Together, these data indicate that BTN3A1 engagement with CD45 and, without wishing to be bound or limited by theory, the short extracellular domain of CD3ζ prevents segregation of CD45 away from the TCR:MHC immune synapse, which is required for effective TCR signaling after LCK^{pY505} dephosphorylation (V. T. Chang et al., Initiation of T cell signaling by CD45 segregation at ‘close contacts’. Nat Immunol 17, 574-582 (2016); Y. Razvag, Y. Neve-Oz, J. Sajman, M. Reches, E. Sherman, Nanoscale kinetic segregation of TCR and CD45 in engaged microvilli facilitates early T cell activation. Nat Commun 9, 732 (2018); J. Irie-Sasaki et al., CD45 is a JAK phosphatase and negatively regulates cytokine receptor signalling. Nature 409, 349-354 (2001); R. R. Hovis et al., Rescue of signaling by a chimeric protein containing the cytoplasmic domain of CD45. Science 260, 544-546 (1993)). Accordingly, these data provide evidence that CD277 is a negative regulator of αβ TCR signaling.

### Example 12

### Anti-BTN3A1 Antibodies Elicit Coordinated αβ and γδ T Cell Responses to Control the Growth of Established Human Ovarian Tumors In Vivo

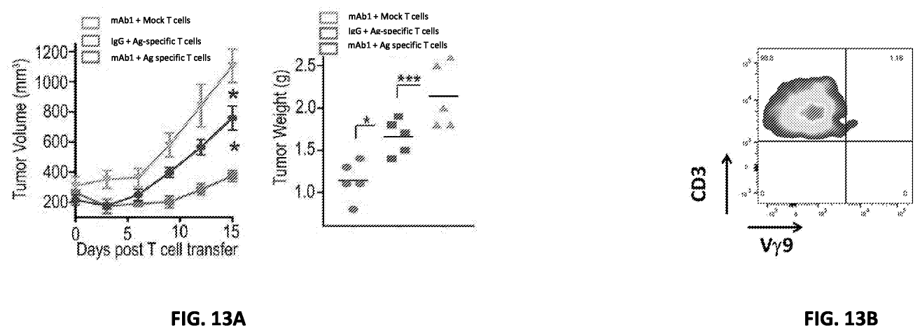

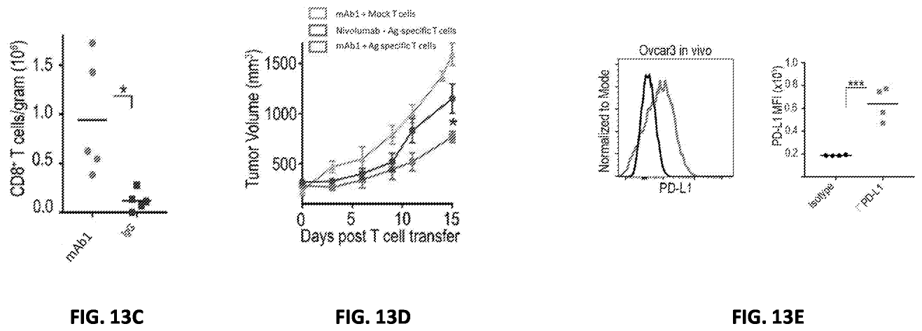

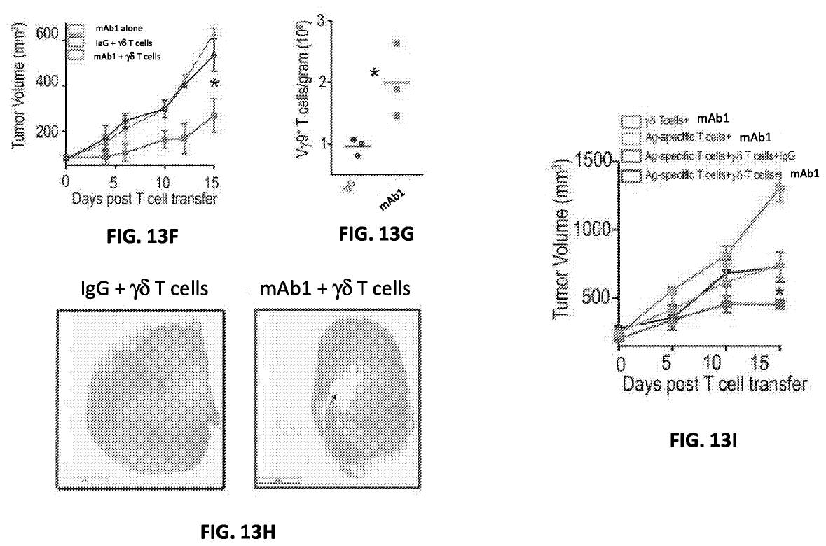

To determine the ability of mAb1 to target BTN3A1 and rescue human αβ T cell anti-tumor responses in vivo, the cancer testis antigen NY-ESO-1 was expressed in HLA-A2⁺BTN3A1⁺ OVCAR3 (NY-OVCAR3) serous ovarian cancer cells, and human αβ T cells were transduced with an HLA A2-restricted TCR that recognizes SLLMWITQC, corresponding to residues 157 to 165 of NY-ESO-1 (P. F. Robbins et al., Tumor regression in patients with metastatic synovial cell sarcoma and melanoma using genetically engineered lymphocytes reactive with NY-ESO-1. J Clin Oncol 29, 917-924 (2011)). To assess effect of mAb1 on the progression of OVCAR3^{NYESO1} tumors, NSG mice were treated with NY-ESO-1 expressing cells or mock-transduced αβ T cells, and intraperitoneally administered mAb1 or control IgG every third day at a dose of 5 mg/kg. As shown in FIGS. 13A and 13B, adoptive transfer of (γδ T cell-free) SLLMWITQC-reactive αβ T cells from seven different donors had significant but modest effects against the growth of established NY-OVCAR3 tumors in nine independent experiments, compared to mock-transduced T cells. Transfer of (γδ T cell-free) SLLMWITQC-reactive αβ T cells in combination with mAb1 administration further reduced tumor growth (FIG. 13A). Remarkably, BTN3A1 blockade with mAb1 delayed malignant progression in every independent experiment to a greater extent that anti-tumor T cells combined with PD-1 blocker NIVOLUMAB® (FIG. 13D), in the presence of PD-L1 upregulation by NY-OVCAR3 tumor cells in vivo (FIG. 13E). Superior therapeutic effects elicited by mAb1 were associated with significant increases in the accumulation of intratumoral IFN-γ-producing antigen-reactive CD8⁺ T cells within OVCAR^{NYESO1} tumors, compared to control IgG-treated mice (FIG. 13C). Tumors in every experiment were allowed to grow for at least 15 days and were treated with a single T cell injection, with significant growth delays for tumors as big as 300 mm³ at the time of adoptive transfer.

The protective effects of mAb1 in vivo were not restricted to tumor-reactive αβ T cells, because γδ T cells from four different donors also elicited tumor growth reduction, albeit only in combination with mAb1 antibodies (FIG. 13F). Accordingly, BTN3A1 targeting resulted in a greater accumulation of Vγ9 T cells within tumor beds (FIG. 13G) (see below), and resulted in the generation of cystic cavities within the tumor (FIG. 13H), indicative of partial tumor rejection. However, maximal anti-tumor responsiveness and the rejection of established tumors was achieved upon co-administration of tumor-specific αβ T cells and autologous γδ T cells (ratio of 6:1) into NY-OVCAR3 tumor-bearing NSG mice, in combination with mAb1 administration from three donors in three experiments (FIG. 13I). Together, these data indicate that targeting BTN3A1 has the potential to orchestrate coordinated responses between tumor-reactive αβ T cells rescued from BTN3A1-mediated immunosuppression and cytotoxic Vγ9Vδ2 T cells re-directed against BTN3A1⁺ tumors.

### Example 13

### Pre-Existing Anti-Tumor Immune Responses in Immunocompetent Hosts are Restored by Anti-CD277 Antibodies

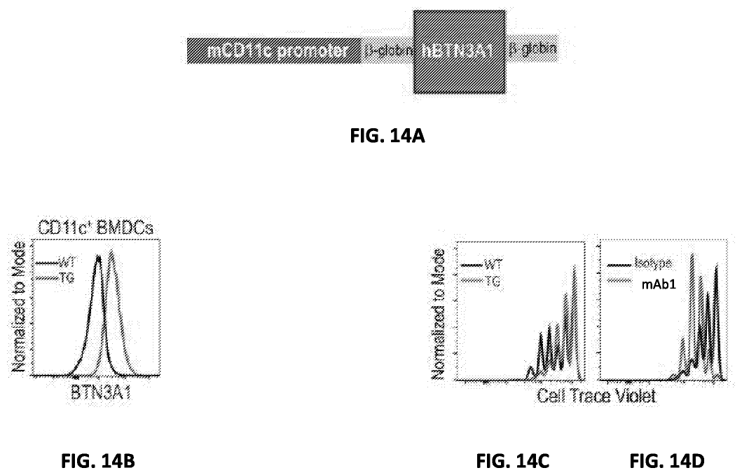

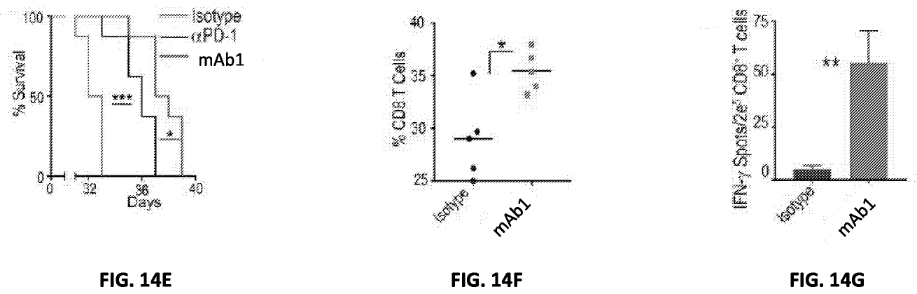

To confirm the anti-tumor effectiveness of blocking BTN3A1 in myeloid cells in immunocompetent hosts, a knock-in mouse expressing human BTN3A1 under the control of the mouse Itgax/Cd11c promoter (BTN3A1^{KI}) (FIG. 14A) was engineered. As expected, CD11c⁺ bone-marrow-dendritic cells (BMDCs) from BTN3A1^{KI} mice expressed human BTN3A1 on the cell surface (FIG. 14B). Equally important, activation of murine T cells in the presence of BTN3A1-Fc fusion proteins decreased IFN-γ release compared to controls, while proliferation of OT-I T cells in response to SIINFEKL-pulsed CD11c⁺BTN3A1⁺ BMDCs was significantly lower than in response to BTN3A1⁻ BMDCs from littermates (FIG. 14C) supporting that human BTN3A1 also functionally suppresses murine αβ T cells. Proliferation of OT-I T cells in the presence of SIINFEKL-pulsed CD11c⁺BTN3A1⁺ BMDCs and anti-CD277 mAb1 rescued mouse T cells from human BTN3A1-mediated suppression (FIG. 14D).

Compared to wild-type controls, BTN3A1^{KI} mice challenged with orthotopic ID8-Defb29/Vegf-a tumors—an aggressive syngeneic ovarian cancer model that responds to checkpoint inhibitors and induces the accumulation of tumor-promoting CD11c⁺ myeloid cells showed accelerated malignant progression (T. L. Stephen et al., SATB1 Expression Governs Epigenetic Repression of PD-1 in Tumor-Reactive T Cells. Immunity 46, 51-64 (2017); A. J. Tesone et al., Satb 1 Overexpression Drives Tumor-Promoting Activities in Cancer-Associated Dendritic Cells. Cell Rep 14, 1774-1786 (2016)). Intraperitoneal administration of mAb1 (100 μg every five days) to BTN3A1KI mice bearing ID8-Defb29/Vegf-a tumors resulted in significantly extended survival (FIG. 14E) when compared to treatment with a PD-1 neutralizing antibody or IgG control. In addition, the accumulation of IFN-γ-producing intratumoral CD8⁺ T cells (FIG. 14F) was increased in mice treated with mAb1. Correspondingly, at day 25 following tumor challenge, the frequencies of peritoneal T cells responding to cognate tumor antigen by secreting IFN-γ in ELISPOT analysis were significantly increased by mAb1 treatment as compared to isotype antibody control (FIG. 14G). As in our humanized model, targeting BTN3A1 was demonstrably more effective in delaying malignant progression than PD-1 neutralization (FIG. 14E). These data therefore underscore the importance of targeting BTN3A1-mediated immunosuppression in the microenvironment of ovarian cancer, a disease so far resistant to existing checkpoint inhibitors.

TABLE 2
Sequence Listing

| SEQ ID | | |
|---|---|---|
| NO | Description | Sequence |
| 1 | Human IgG1 | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISR |
| | | TPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| 2 | CD277 | MKMASFLAFLLLNFRVCLLLLQLLMPHSAQFSVLGPSGPILAMVGE |
| | | DADLPCHLFPTMSAETMELKWVSSSLRQVVNVYADGKEVEDRQSAP |
| | | YRGRTSILRDGITAGKAALRIHNVTASDSGKYLCYFQDGDFYEKAL |
| | | VELKVAALGSDLHVDVKGYKDGGIHLECRSTGWYPQPQIQWSNNKG |
| | | ENIPTVEAPVVADGVGLYAVAASVIMRGSSGEGVSCTIRSSLLGLE |
| | | KTASISIADPFFRSAQRWIAALAGTLPVLLLLLGGAGYFLWQQQEE |
| | | KKTQFRKKKREQELREMAWSTMKQEQSTRVKLLEELRWRSIQYASR |
| | | GERHSAYNEWKKALFKPADVILDPKTANPILLVSEDQRSVQRAKEP |
| | | QDLPDNPERFNWHYCVLGCESFISGRHYWEVEVGDRKEWHIGVCSK |
| | | NVQRKGWVKMTPENGFWTMGLTDGNKYRTLTEPRTNLKLPKPPKKV |
| | | GVFLDYETGDISFYNAVDGSHIHTFLDVSFSEALYPVFRILTLEPT |
| | | ALTICPA |
| 3 | V_{H1} amino acid | QVQLVQSGAEVKKPGASVKVSCKASGYTFTGYYMHWVRQAPGQGLE |
| | sequence | WMGWINPNSGGTKYAQKFQGRVTMTRDTSISTAYMELSRLRSDDTA |
| | | VYYCARRHSDMIGYYYGMDVWGQGTTVTVSS |
| 4 | V_{L1} amino acid | DIQMTQSPSSVSASVGDRVTITCRASQGISSWLAWYQQKPGKAPKL |
| | sequence | IYAASSLQSGVPSRFSGSGSGTDFTLTISSLQPEDFATYYCQQATD |
| | | FPPTFGGGTKVEIK |
| 5 | V_{H1} nucleic acid | CAAGTCCAGCTTGTGCAATCCGGGGCAGAGGTTAAAAAGCCCGGGG |
| | sequence | CTTCCGTCAAGGTATCATGTAAGGCTTCAGGATATACATTCACAGG |
| | | GTACTATATGCACTGGGTGCGTCAGGCACCCGGGCAGGGCTTGGAA |
| | | TGGATGGGGTGGATAAACCCAAATAGTGGAGGCACTAAATATGCTC |
| | | AAAAGTTCCAAGGGCGGGTGACTATGACCAGGGACACCAGTATCTC |
| | | CACCGCCTATATGGAACTGTCACGACTCAGATCAGACGATACCGCT |
| | | GTATATTACTGCGCTCGTAGACACTCAGACATGATTGGATACTACT |
| | | ATGGAATGGACGTATGGGGCCAAGGGACTACAGTTACAGTCTCTAG |
| | | C |
| 6 | V_{L1} nucleic acid | GACATACAGATGACACAAAGCCCAAGCAGCGTCAGCGCAAGTGTCG |
| | sequence | GTGACCGCGTCACAATAACTTGTCGGGCTAGTCAAGGAATAAGCTC |
| | | TTGGCTCGCCTGGTATCAACAAAAACCTGGCAAAGCACCCAAGCTG |
| | | TTGATCTACGCAGCCAGCTCACTTCAGAGCGGAGTGCCCAGTCGCT |
| | | TCTCTGGTTCCGGCTCAGGTACTGATTTCACACTTACTATTTCATC |
| | | ACTGCAACCCGAGGATTTCGCAACATATTACTGTCAACAGGCCACA |
| | | GACTTTCCACCAACTTTTGGTGGAGGCACAAAGGTCGAAATTAA |
| 7 | HCDR1 | YTFTGYYMH |
| 8 | HCDR2 | WINPNSGGTKYAQKFQG |
| 9 | HCDR3 | ARRHSDMIGYYYGMDV |
| 10 | LCDR1 | RASQGISSWLA |
| 11 | LCDR2 | AASSLQS |
| 12 | LCDR3 | QQATDFPPT |
| 13 | CD277 | QFSVLGPSGPILAMVGEDADLPCHLFPTMSAETMELKWVSSSLRQV |
| | Extracellular | VNVYADGKEVEDRQSAPYRGRTSILRDGITAGKAALRIHNVTASDS |
| | Domain | GKYLCYFQDGDFYEKALVELKVAALGSDLHVDVKGYKDGGIHLECR |
| | | STGWYPQPQIQWSNNKGENIPTVEAPVVADGVGLYAVAASVIMRGS |
| | | SGEGVSCTIRSSLLGLEKTASISIADPFFRSAQRWIAALAG |
| 14 | FLAG | DYKDDDDK |
| 15 | Polyhistidine (6- | HHHHHH |
| | His) | |
| 16 | Hemagglutinin | YPYDVPDYA |
| | (HA) | |
| 17 | Human IgG4 | ASTKGPSVFPLAPCSRSTSESTAALGCLVKDYFPEPVTVSWNSGAL |
| | mutant (S228P/C- | TSGVHTFPAVLQSSGLYSLSSVVTVPSSSLGTKTYTCNVDHKPSNT |
| | terminal K | KVDKRVESKYGPPCPPCPAPEFLGGPSVFLFPPKPKDTLMISRTPE |
| | truncation) | VTCVVVDVSQEDPEVQFNWYVDGVEVHNAKTKPREEQFNSTYRVVS |
| | | VLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPP |
| | | VLDSDGSFFLYSRLTVDKSRWQEGNVFSCSVMHEALHNHYTQKSLS |
| | | LSLG |
| 18 | VHFR1 | QVQLVQSGAEVKKPGASVKVSCKASG |
| 19 | VHFR2 | WVRQAPGQGLEWMG |
| 20 | VHFR3 | RVTMTRDTSISTAYMELSRLRSDDTAVYYC |
| 21 | VHFR4 | WGQGTTVTVSS |
| 22 | VLFR1 | DIQMTQSPSSVSASVGDRVTITC |
| 23 | VLFR2 | WYQQKPGKAPKLLIY |
| 24 | VLFR3 | GVPSRFSGSGSGTDFTLTISSLQPEDFATYYC |
| 25 | VLFR4 | FGGGTKVEIK |
| 26 | mAb1 heavy | QVQLVQSGAEVKKPGASVKVSCKASGYTFTGYYMHWVRQAPGQGLE |
| | chain | WMGWINPNSGGTKYAQKFQGRVTMTRDTSISTAYMELSRLRSDDTA |
| | | VYYCARRHSDMIGYYYGMDVWGQGTTVTVSSASTKGPSVFPLAPSS |
| | | KSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTH |
| | | TCPPCPAPELLGGPSVFLFPPKPKDTLMISRTPEVTCVVVDVSHED |
| | | PEVKFNWYVDGVEVHNAKTKPREEQYASTYRVVSVLTVLHQDWLNG |
| | | KEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPG |
| 27 | mAb1 light | DIQMTQSPSSVSASVGDRVTITCRASQGISSWLAWYQQKPGKAPKL |
| | chain | LIYAASSLQSGVPSRFSGSGSGTDFTLTISSLQPEDFATYYCQQAT |
| | | DFPPTFGGGTKVEIKRTVAAPSVFIFPPSDEQLKSGTASVVCLLNN |
| | | FYPREAKVQWKVDNALQSGNSQESVTEQDSKDSTYSLSSTLTLSKA |
| | | DYEKHKVYACEVTHQGLSSPVTKSFNRGEC |
| 28 | | SLLMWITQC |
| 29 | crRNA | UUACCACAUGUUGGCUUAGA |
| 30 | crRNA | AGUACAUGAAUUAUGAGAUA |
| 31 | HCDR2.1 | GWINPNSGGTKYA |
| 32 | VHFR2.1 | WVRQAPGQGLEWM |
| 33 | VHFR3.1 | QKFQGRVTMTRDTSISTAYMELSRLRSDDTAVYYC |
| 34 | V_{L1-2} amino acid | DIQMTQSPSSVSASVGDRVTITCRASQGISSWLAWYQQKPGKAPKL |
| | sequence | LIYAASSLQSGVPSRFSGSGSGTDFTLTISSLQPEDFATYYCQQAT |
| | | DFPPTFGGGTKVEIK |

1. A method for inducing or enhancing CD277-mediated γδ T cell stimulation in a subject, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal.

2. The method of claim 1, wherein the CD277-mediated γδ T cell stimulation is CD277-mediated γδ T cell proliferation.

3. The method of any one of claim 1 or 2, wherein the CD277-mediated γδ T cell stimulation is CD277-mediated cytokine production by a γδ T cell.

4. The method of claim 3, wherein the cytokine production is IFNγ production.

5. A method for reducing CD277-mediated inhibition of αβ T cells in a subject, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal.

6. The method of claim 5, wherein the reduction of CD277-mediated inhibition of αβ T cells is CD277-mediated αβ T cell proliferation.

7. The method of any one of claims 5-6, wherein the reduction of CD277-mediated inhibition of αβ T cells is CD277-mediated cytokine production by a αβ T cell.

8. The method of claim 7, wherein the cytokine production is IFNγ production.

9. The method of any one of claims 1-8, wherein the costimulatory signal results from CD3 engagement or CD28 engagement.

10. A method for reducing or inhibiting tumor growth, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal.

11. A method for treating cancer in a subject, comprising administering to a subject in need thereof, an effective amount of an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal.

12. A method for reducing or inhibiting tumor growth, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal.

13. A method for treating cancer in a subject, comprising administering to a subject in need thereof, an effective amount of an isolated monoclonal antibody that specifically binds human CD277, or an antigen-binding portion thereof, wherein the antibody or antigen-binding fragment thereof reduces CD277-mediated inhibition of αβ T cells in the absence of one or both of: (i) a phosphoantigen and (ii) a costimulatory signal.

14. The method of any one of claims 1-13, wherein the antibody or antigen binding portion thereof comprises heavy chain CDR1, CDR2 and CDR3 sequences set forth in SEQ ID NOs: 7-9, respectively, and light chain CDR1, CDR2 and CDR3 sequences set forth in SEQ ID NOs: 10-12, respectively.

15. The method of any one of claims 1-13, wherein the antibody or antigen binding portion thereof comprises heavy and light chain variable regions comprising amino acid sequences set forth in SEQ ID NOs: 3 and 4, respectively.

16. The method of any one of claims 1-13, wherein the antibody or antigen binding portion thereof comprises heavy and light chain variable regions comprising amino acid sequences at least 90% identical to the amino acid sequences set forth in SEQ ID NOs: 3 and 4, respectively.

17. The method of any one of claims 1-13, wherein the antibody comprises heavy and light chains comprising amino acid sequences set forth in SEQ ID NOs: 26 and 27, respectively.

18. The method of any one of claims 1-13, wherein the antibody comprises heavy and light chains comprising amino acid sequences at least 90% identical to the amino acid sequences set forth in SEQ ID NOs: 26 and 27, respectively.

19. The method of any one of claims 1-18, wherein the antibody or antigen-binding portion thereof is chimeric or humanized.

20. The method of any one of claims 1-18, wherein the antibody or antigen-binding portion thereof is a fully human antibody or antigen-binding portion thereof.

21. The method of any one of claims 1-20, wherein the antibody or antigen-binding portion thereof binds to cynomolgus macaque CD277.

22. The method of any one of claims 1-21, wherein the antibody is selected from the group consisting of an IgG1, an IgG2, and IgG3, an IgG4, and IgM, and IgA1, and IgA2, and IgD, and an IgE antibody.

23. The method of claim 22, wherein the antibody is an IgG1 antibody or IgG4 antibody.

24. A composition comprising an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal, for use in treating or delaying progression of a cancer, or reducing or inhibiting tumor growth, in a subject in need thereof.

25. Use of a composition comprising an isolated monoclonal antibody that specifically binds human CD277, or an antigen-binding portion thereof, wherein the antibody or antigen-binding fragment thereof induces or enhances CD277-mediated γδ T cell stimulation in the absence of one or both of: (i) a phosphoantigen and (ii) a costimulatory signal, and a pharmaceutically acceptable carrier, in the manufacture of a medicament for treating or delaying progression of a cancer, or reducing or inhibiting tumor growth, in a subject in need thereof.

26. The composition or use of the composition of any one of claims 24-25, wherein the antibody or antigen binding portion thereof comprises heavy chain CDR1, CDR2 and CDR3 sequences set forth in SEQ ID NOs: 7-9, respectively, and light chain CDR1, CDR2 and CDR3 sequences set forth in SEQ ID NOs: 10-12, respectively.

27. The composition or use of the composition of any one of claims 24-26, wherein the antibody or antigen binding portion thereof comprises heavy and light chain variable regions comprising amino acid sequences set forth in SEQ ID NOs: 3 and 4, respectively.

28. The composition or use of the composition of any one of claims 24-26, wherein the antibody or antigen binding portion thereof comprises heavy and light chain variable regions comprising amino acid sequences at least 90% identical to the amino acid sequences set forth in SEQ ID NOs: 3 and 4, respectively.

29. The composition or use of the composition of any one of claims 24-26, wherein the antibody comprises heavy and light chains comprising amino acid sequences set forth in SEQ ID NOs: 26 and 27, respectively.

30. The composition or use of the composition of any one of claims 24-26, wherein the antibody comprises heavy and light chains comprising amino acid sequences at least 90% identical to the amino acid sequences set forth in SEQ ID NOs: 26 and 27, respectively.

31. The composition or use of the composition of any one of claims 24-30, wherein the antibody or antigen-binding portion thereof is chimeric or humanized.

32. The composition or use of the composition of any one of claims 24-30, wherein the antibody or antigen-binding portion thereof is a fully human antibody or antigen-binding portion thereof.

33. The composition or use of the composition of any one of claims 24-32, wherein the antibody or antigen-binding portion thereof binds to cynomolgus macaque CD277.

34. The composition or use of the composition of any one of claims 24-33, wherein the antibody is selected from the group consisting of an IgG1, an IgG2, and IgG3, an IgG4, and IgM, and IgA1, and IgA2, and IgD, and an IgE antibody.

35. The composition or use of the composition of claim 34, wherein the antibody is an IgG1 antibody or IgG4 antibody.

36. An antigen-binding agent that specifically binds human CD277, wherein the antigen-binding agent binds to human CD277 and inhibits the interaction of CD277 with CD45, thereby activating or enhancing an αβ T cell response.

37. An antigen-binding agent that specifically binds human CD277, wherein the antigen-binding agent binds to human CD277 and inhibits the interaction of CD277 with CD45, thereby disinhibiting the immunosuppressive effect of CD277 toward αβ T cells.

38. An antigen-binding agent that specifically binds human CD277, wherein the antigen-binding agent binds to human CD277 and inhibits CD277-mediated association of CD45 with CD3ζ, thereby activating or enhancing an αβ T cell response.

39. An antigen-binding agent that specifically bind human CD277 wherein the antigen-binding agent binds to human CD277 and inhibits CD277-mediated association of CD45 with the TCR/MHC immune synapse, thereby activating or enhancing an αβ T cell response.

40. An antigen-binding agent that specifically bind human CD277 wherein the antigen-binding agent binds to human CD277 and increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent, or antigen-binding portion thereof, thereby activating or enhancing an αβ T cell response.

41. The antigen-binding agent of any one of claims 36-40, wherein the antigen-binding agent, or antigen-binding portion thereof, is selected from the group consisting of an antibody, a non-antibody scaffold protein, a polypeptide, a small molecule, and a nucleic acid.

42. The antigen-binding agent of any one of claims 36-41, wherein the antigen-binding agent, or antigen-binding portion thereof, further induces or enhances γδ T cell response.

43. The antigen-binding agent of claim 40, wherein the TCR signaling molecules are phosphorylated at activating residues LCK^{pY394}, Zap70^{pY319}, and CD3ζ^{pY142}.

44. The antigen-binding agent of claim 40, wherein the activating residue is LCK^{pY394}.

45. The antigen-binding agent of claim 40, wherein the activating residue is Zap70^{pY319}.

46. The antigen-binding agent of claim 40, wherein the activating residue is CD3ζ^{pY142}.

47. The antigen-binding agent of any one of claims 36-46, wherein the antigen-binding agent is a monoclonal antibody or antigen-binding portion thereof.

48. The antigen-binding agent of claim 47, wherein the antibody or antigen-binding portion thereof is chimeric or humanized.

49. The antigen-binding agent of claim 47, wherein the antibody or antigen-binding portion thereof is a fully human antibody or antigen-binding portion thereof.

50. The antigen-binding agent of any one of claims 47-49, wherein the antibody is selected from the group consisting of an IgG1, an IgG2, and IgG3, an IgG4, and IgM, and IgA1, and IgA2, and IgD, and an IgE antibody.

51. The antigen-binding agent of claim 50, wherein the antibody is an IgG1 antibody or IgG4 antibody.

52. A composition comprising the monoclonal antibody or antigen-binding portion thereof, of any one of claims 47-51, and a pharmaceutically acceptable carrier.

53. A nucleic acid comprising a nucleotide sequence encoding the light chain, heavy chain, or both light and heavy chains of the monoclonal antibody, or antigen-binding portion thereof, of any one of claims 47-51.

54. An expression vector comprising the nucleic acid of claim 53.

55. A cell transformed with the expression vector of claim 54.

56. A method for treating cancer in a subject, comprising administering to a subject in need thereof, an effective amount of the antigen-binding agent, or antigen-binding portion thereof, of any one of claims 36-42.

57. The method of claim 56, wherein the cancer is a solid tumor.

58. The method of claim 56, wherein the cancer is ovarian cancer.

59. Use of a composition comprising the antigen-binding agent, or antigen-binding portion thereof, of any one of claims 36-42, and a pharmaceutically acceptable carrier, in the manufacture of a medicament for treating or delaying the progression of cancer, or reducing or inhibiting tumor growth in a subject in need thereof.

60. The use of claim 59, wherein the cancer is a solid tumor.

61. The use of claim 59, wherein the cancer is ovarian cancer.

62. A composition comprising the antigen-binding agent, or antigen-binding portion thereof, of any one of claims 36-42, for use in treating or delaying progression of a cancer, or reducing or inhibiting tumor growth, in a subject in need thereof.

63. The composition of claim 62, wherein the cancer is a solid tumor.

64. The composition of claim 62, wherein the cancer is ovarian cancer.

65. A method for identifying an antigen-binding agent of interest, the method comprising:
(a) contacting, in the presence of a CD45 protein, a CD277 protein with a test antigen-binding agent; and
(b) identifying the test antigen-binding agent as an antigen-binding agent of interest if the test antigen-binding agent inhibits the interaction between CD45 and CD277.

66. The method of claim 65, wherein one or both of the CD277 protein and the CD45 protein are recombinant proteins.

67. The method of claim 65, wherein one or both of the CD277 protein and the CD45 protein is expressed on the surface of cells.

68. The method of claim 67, wherein the CD45 protein is expressed on an αβ T cell.

69. A method for identifying an antigen-binding agent of interest, the method comprising:
(a) contacting, in the presence of a CD45 protein, a CD277 protein with a test antigen-binding agent, wherein the CD45 protein is expressed by an αβ T cell; and
(b) identifying the test antigen-binding agent as an antigen-binding agent of interest if the test antigen-binding agent increases the level of phosphorylation of one or more TCR signaling molecules at activating residues selected from the group consisting of LCK^{pY394}, Zap70^{pY319}, and CD3ζ^{pY142}, relative to the level of phosphorylation of the one or more TCR signaling molecules in the absence of the antigen-binding agent.

70. A method for detecting the immunomodulatory activity of an antigen-binding agent, the method comprising:
(a) detecting the presence, absence, or amount of association of: (i) CD45 with CD3ζ or (ii) CD45 with the TCR/MHC immune synapse on one or more αβ T cells from a subject administered an antigen-binding agent according to any one of claims 1-15, wherein an increase in the association of (i) CD45 with CD3ζ or (ii) CD45 with the TCR/MHC immune synapse on the one or more αβ T cells relative to a control level of association indicates that the antigen-binding agent has immunomodulatory activity.

71. A method for detecting the immunomodulatory activity of an antigen-binding agent, the method comprising:
(a) detecting the presence, absence, level, or amount of phosphorylation of one or more TCR signaling molecules at activating residues selected from the group consisting of LCK^{pY394}, Zap70^{pY319}, and CD3ζ^{pY142} by one or more αβ T cells from a subject administered an antigen-binding agent according to any one of claims 1-15, wherein an increase in the level or amount of phosphorylation of one or more TCR signaling molecules at activating residues selected from the group consisting of LCK^{pY394}, Zap70^{pY319}, and CD3ζ^{pY142} by one or more αβ T cells relative to a control level or amount of phosphorylation, indicates that the antigen-binding agent has immunomodulatory activity.

72. The method of claim 70 or 71, further comprising obtaining the one or more T cells from the subject.

73. A method for treating cancer in a subject, the method comprising administering to the subject the antigen-binding agent according to any one of claims 36-51 in an amount effective to treat the cancer, wherein the antigen-binding agent has been determined to have an immunomodulatory effect in the patient.

74. The method of claim 73, wherein the immunomodulatory effect was determined according to claim 70 or 71.

75. The antigen-binding agent of any one of claims 36-51, the composition of any one of claim 52, or 62-64, the nucleic acid of claim 53, the expression vector of claim 54, the cell of claim 55, method of any one of claim 56-58 or 65-74, or use of claims 59-61, wherein the antigen-binding agent comprises heavy chain CDR1, CDR2, and CDR3 sequences set forth in SEQ ID NOs: 7-9, respectively, and light chain CDR1, CDR2, and CDR3 sequences set forth in SEQ ID NOs: 10-12, respectively.

76. The antigen-binding agent of any one of claims 36-51, the composition of any one of claim 52, or 62-64, the nucleic acid of claim 53, the expression vector of claim 54, the cell of claim 55, method of any one of claim 56-58 or 65-74, or use of claims 59-61, wherein the antigen-binding agent comprises a heavy chain variable region having at least 90% identity to SEQ ID NO: 3 and a light chain variable region having at least 90% identity to SEQ ID NO: 4.

77. The antigen-binding agent of any one of claims 36-51, the composition of any one of claim 52, or 62-64, the nucleic acid of claim 53, the expression vector of claim 54, the cell of claim 55, method of any one of claim 56-58 or 65-74, or use of claims 59-61, wherein the antigen-binding agent comprises heavy chain CDR1, CDR2, and CDR3 sequences set forth in SEQ ID NOs: 7, 31, and 9, respectively, and light chain CDR1, CDR2, and CDR3 sequences set forth in SEQ ID NOs: 10-12, respectively.

78. The antigen-binding agent of any one of claims 36-51, the composition of any one of claim 52, or 62-64, the nucleic acid of claim 53, the expression vector of claim 54, the cell of claim 55, method of any one of claim 56-58 or 65-74, or use of claims 59-61, wherein the antigen-binding agent comprises a heavy chain variable region having at least 90% identity to SEQ ID NO: 3 and a light chain variable region having at least 90% identity to SEQ ID NO: 34.

79. The composition or use of the composition of any one of claims 24-25, wherein the antibody or antigen binding portion thereof comprises heavy chain CDR1, CDR2 and CDR3 sequences set forth in SEQ ID NOs: 7, 31, and 9, respectively, and light chain CDR1, CDR2 and CDR3 sequences set forth in SEQ ID NOs: 10-12, respectively.

80. The composition or use of the composition of any one of claims 24-26, wherein the antibody or antigen binding portion thereof comprises heavy and light chain variable regions comprising amino acid sequences set forth in SEQ ID NOs: 3 and 34, respectively.

81. The composition or use of the composition of any one of claims 24-26, wherein the antibody or antigen binding portion thereof comprises heavy and light chain variable regions comprising amino acid sequences at least 90% identical to the amino acid sequences set forth in SEQ ID NOs: 3 and 34, respectively.
